(19) United States
(12) Patent Application Publication
(10) Pub. No.: US 2014/0107049 A1
(43) Pub. Date: Apr. 17, 2014

(54) MANNOSE DERIVATIVES FOR TREATING BACTERIAL INFECTIONS

(71) Applicant: Vertex Pharmaceuticals Incorporated, Cambridge, MA (US)

(72) Inventors: Youssef Laafiret Bennani, Lorraine (CA); Bingcan Liu, Montreal (CA)

(73) Assignee: Vertex Pharmaceuticals Incorporated, Cambridge, MA (US)

(21) Appl. No.: 14/042,817

(22) Filed: Oct. 1, 2013

Related U.S. Application Data
(60) Provisional application No. 61/709,686, filed on Oct. 4, 2012.

(51) Int. Cl. C07H 15/20; C07H 15/26; C07H 15/04

(52) U.S. Cl. CPC C07H 15/20; C07H 15/04; C07H 15/26
USPC 514/27; 536/16.8; 514/25; 435/375

(57) ABSTRACT

The present invention relates to compounds useful for the treatment or prevention of bacteria infections. The invention also provides pharmaceutically acceptable compositions containing the compounds and methods of using the compositions in the treatment of bacteria infections. The invention also provides processes for making the compounds of the invention.

The compounds of the present invention are represented by the following structure of Formula A:

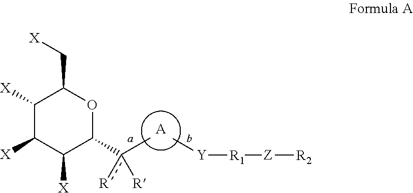

wherein the variables are as described herein.

### CROSS REFERENCE TO RELATED APPLICATIONS

This present invention claims the benefit, under 35 U.S.C. §119, of U.S. Provisional Application No. 61/709,686, filed Oct. 4, 2012; the entire contents of which are incorporated herein by reference.

### BACKGROUND OF THE INVENTION

Inflammatory bowel disease (IBD) is a complex chronic inflammatory disorder, with the two more common forms being ulcerative colitis (UC) and Crohn's disease (CD). IBD is a multifactorial disease that results from a combination of predisposing genetic factors, environmental triggers, dysbiosis of the gastrointestinal microbiota and an inappropriate inflammatory response (Man et al., 2011, Nat Rev Gastroenterol Hepatol, March, 8(3):152-68).

Several studies on fecal and mucosa-associated bacterial communities have shown that the microbiota of patients with Crohn's disease (CD) differ from those of healthy controls, as well as those of patients with ulcerative colitis (UC). Although the reported changes are not always consistent, numbers of Escherichia coli are generally increased, whereas Firmicutes are scarcer in CD patients (Peterson et al., 2008, Cell Host Microbe, 3: 17-27; Frank et al., 2007, Proc. Natl. Acad. Sci., 104:13780-13785). Whether these changes are causative factors or consequences of inflammation, it remains controversial. To date, several pathogens have been proposed as causative agents. In particular, adherent-invasive E. coli (AIEC) has been reported to be more prevalent in CD patients than in controls in several countries (United Kingdom, France and the USA) (Darfeuille-Michaud et al., 2004, Gastroenterology, 127:412-421; Martinez-Medina et al., 2009, Inflamm Bowel Dis., 15:872-882). AIEC strains have been isolated from ileal lesions in ˜35% of CD patients compared to ˜5% of healthy subjects. One of the features of AIEC is their ability to adhere and invade epithelial cells. It is known from various models that the binding of adhesins expressed on the bacterial cell surface to defined glycosylated receptors on the host tissue surface is considered to be an initial and critical step in pathogenesis, then opening a new avenue for therapy such as blocking the interaction between type 1 pili and CEACAM6, a known host receptor for FimH (Barnich et al., 2007, J. Clin. Invest., 117:1566-1574; Carvalho et al., 2009, JEM, vol. 206, no. 10, 2179-2189). Therefore, inhibition of adhesion, and consequently intracellular replication of AIEC in epithelial cells, may prevent establishment of a sub-mucosal infection leading to mucosal inflammation and epithelial barrier disruption.

It has also been demonstrated recently that FimH antagonists are potentially effective in treating urinary tract infections (J. Med. Chem. 2010, 53, 8627-8641).

### SUMMARY OF THE INVENTION

The present invention provides compounds useful for the treatment or prevention of bacteria infections, such as urinary tract infection (UTI) and inflammatory bowel diseases (IBD). The compounds of the present invention are represented by the following structure of Formula A:

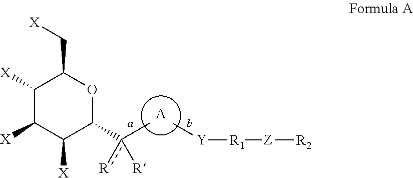

wherein the variables are as described herein.

The present invention also provides a composition comprising the compound described herein, and a pharmaceutically acceptable carrier, adjuvant, or vehicle.

The present invention also provides a method of treating or preventing bacteria infection in a subject, comprising administering to the subject an effective amount of the compound or the composition described herein.

The present invention also provides processes for making the compounds of the invention.

### DETAILED DESCRIPTION OF THE INVENTION

One aspect of the invention provides a compound of Formula A:

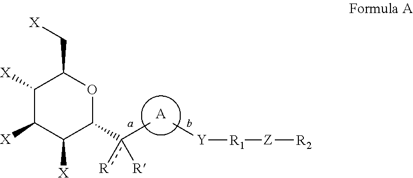

wherein:
Ring A is

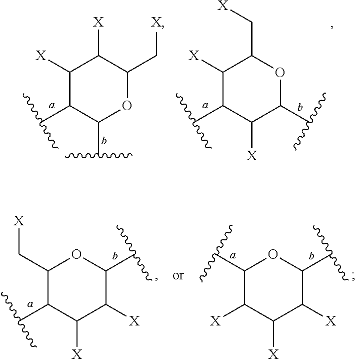

each X is independently —H, halogen, (C₁-C₆)alkyl, —NR₅R₆, —SR₇, or —OR₇;
Y and Z are each independently absent, —NR₈, —O—, or —S—;
R′ is absent, —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups;
R is —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups; wherein the dashed line represents a second bond which may be present or absent, and when present R is ═O, ═NOR₄, or ═C(R₄)₂, and R′ is absent; or
R and R′ together form a cyclic ring or a heterocyclic ring containing from 1 to 3 heteroatoms; each optionally substituted with one or more R₃ groups;
R₁ is alkyl, alkenyl, alkynyl, cycloalkyl, heterocyclyl, aryl, or heteroaryl; each optionally substituted with one or more R₃ groups;
R₂ is —H, or alkyl, alkenyl, alkynyl, cycloalkyl, heterocyclyl, aryl or heteroaryl; each optionally substituted with one or more R₃ groups, or -M-Q;
M is —C(O)O—, —C(O)—, —C(O)N(R₈)(CH₂)ₙ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈—, or (C₁-C₆)alkyl, (C₁-C₆)alkenyl or (C₁-C₆)alkynyl, wherein said (C₁-C₆)alkyl, (C₁-C₆)alkenyl or (C₁-C₆)alkynyl is optionally substituted with one or more R₃ groups;
Q is cycloalkyl, heterocyclyl, aryl or heteroaryl optionally substituted with one or more R₃ groups;
R₃ is —OH, oxo, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —C(O)NHR₄, —R₄—C(O)N(R₄)₂, —R₄—C(O)NHR₄, —N(R₄)C(O)(R₄), —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; wherein R₃ is optionally substituted with one or more R₄; and wherein each alkyl, alkenyl, alkynyl, cycloalkyl, heterocyclo, aryl, aralkyl, or heteroaryl is further optionally substituted with one or more OH or NR₂;
R₄ is —H, or optionally substituted C₁-C₆ alkyl, C₁-C₆ alkenyl, C₁-C₆ alkynyl, cycloalkyl, heterocyclyl, aryl or heteroaryl;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, —C(O)R₉, or —C(O)NHR₉, or optionally substituted alkyl, alkenyl, alkynyl, cycloalkyl, heterocyclyl, heteroaryl or aryl;
R₈ is —H, —C(O)R₉, or optionally substituted alkyl, alkenyl, alkynyl, cycloalkyl, heterocyclyl, heteroaryl, or aryl;
R₉ is —H, optionally substituted alkyl, cycloalkyl, heterocyclyl, aryl or heteroaryl;
R₁₀ is —H, halogen, or optionally substituted C₁-C₆ alkyl, C₁-C₆ alkenyl, C₁-C₆ alkynyl, cycloalkyl, heterocyclyl, aryl or heteroaryl; and
n is 0, 1, 2, 3 or 4.

Another aspect of the invention provides a compound of Formula (I), (II), (III), or (IV), or a pharmaceutically acceptable salt thereof:

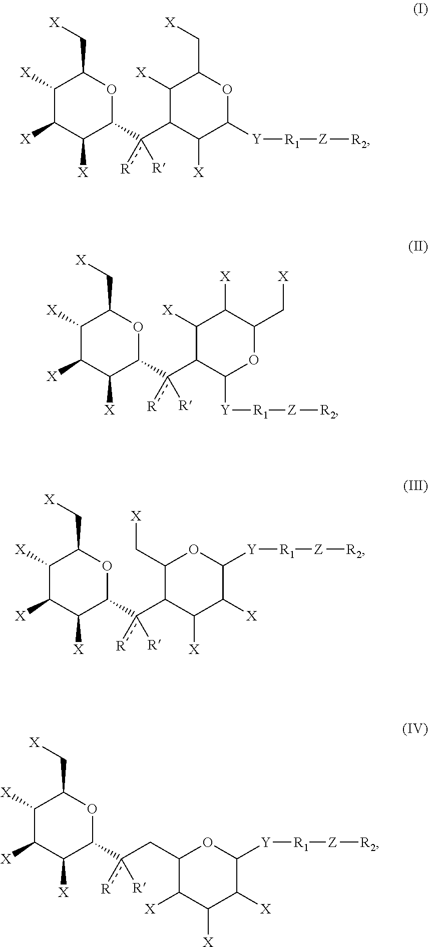

wherein:
each X is independently —H, halogen, (C₁-C₆)alkyl, —NR₅R₆, —SR₇, or —OR₇;
Y and Z are each independently absent, —NR₈, —O—, or —S—;
R′ is absent, —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups;
R is —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups; wherein the dashed line represents a second bond which may be present or absent, and when present R is ═O, ═NOR₄, or ═C(R₄)₂, and R′ is absent; or
R and R′ together form a cyclic ring or a heterocyclic ring containing from 1 to 3 heteroatoms; each optionally substituted with one or more R₃ groups;
R₁ is alkyl, alkenyl, alkynyl, cycloalkyl, heterocyclyl, aryl, or heteroaryl; each optionally substituted with one or more R₃ groups;
R₂ is —H, or alkyl, alkenyl, alkynyl, cycloalkyl, heterocyclyl, aryl or heteroaryl; each optionally substituted with one or more R₃ groups, or -M-Q;
M is —C(O)O—, —C(O)—, —C(O)N(R₈)(CH₂)ₙ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈—, or (C₁-C₆)alkyl, (C₁-C₆)alkenyl or (C₁-C₆)alkynyl, wherein said (C₁-C₆)alkyl, (C₁-C₆)alkenyl or (C₁-C₆)alkynyl is optionally substituted with one or more R₃ groups;
Q is cycloalkyl, heterocyclyl, aryl or heteroaryl optionally substituted with one or more R₃ groups;
R₃ is —OH, oxo, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —C(O)NHR₄, —R₄—C(O)N(R₄)₂, —R₄—C(O)NHR₄, —N(R₄)C(O)(R₄), —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; wherein R₃ is optionally substituted with one or more R₄; and wherein each alkyl, alkenyl, alkynyl, cycloalkyl, heterocyclo, aryl, aralkyl, or heteroaryl is further optionally substituted with one or more OH or NR₇;
R₄ is —H, or optionally substituted C₁-C₆ alkyl, C₁-C₆ alkenyl, C₁-C₆ alkynyl, cycloalkyl, heterocyclyl, aryl or heteroaryl;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, —C(O)R₉, or —C(O)NHR₉, or optionally substituted alkyl, alkenyl, alkynyl, cycloalkyl, heterocyclyl, heteroaryl or aryl;
R₈ is —H, —C(O)R₉, or optionally substituted alkyl, alkenyl, alkynyl, cycloalkyl, heterocyclyl, heteroaryl, or aryl;
R₉ is —H, optionally substituted alkyl, cycloalkyl, heterocyclyl, aryl or heteroaryl;
R₁₀ is —H, halogen, or optionally substituted C₁-C₆ alkyl, C₁-C₆ alkenyl, C₁-C₆ alkynyl, cycloalkyl, heterocyclyl, aryl or heteroaryl; and
n is 0, 1, 2, 3 or 4.

In some embodiments, R₃ is —OH, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —C(O)NHR₄, —R₄—C(O)N(R₄)₂, —R₄—C(O)NHR₄, —N(R₄)C(O)(R₄), —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; wherein R₃ is optionally substituted with one or more R₄; and wherein each alkyl, alkenyl, alkynyl, cycloalkyl, heterocyclo, aryl, aralkyl, or heteroaryl is further optionally substituted with one or more OH or NR₇;

In some embodiments, the compound has the structure of Formula (I), or a pharmaceutically acceptable salt thereof:

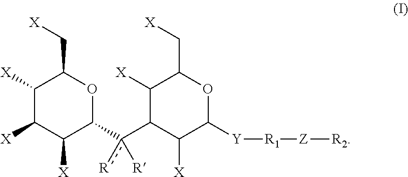

In other embodiments, the compound the Formula (I) has the following structure:

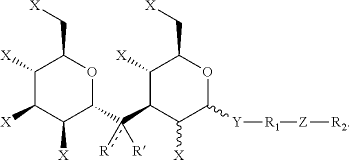

In yet other embodiments, the Formula (I) has the following structure:

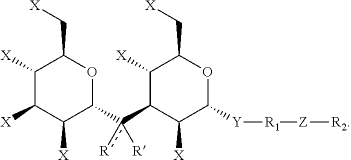

In some embodiments, each X is independently —OH, —F, —OCH₃, or —CH₃. In other embodiments, X is —OH.

In some embodiments,
Y is —O—;
Z is absent;
R is —H or OH and the dashed line representing the second bond is absent;
R′ is —H;
R₁ is phenyl or benzo[d][1,3]dioxolyl optionally substituted with one R₃ group;
R₂ is —H, phenyl, or a 5-6 membered heterocyclyl or heteroaryl ring containing 1 to 2 nitrogen atoms; each optionally substituted with one or more R₃ groups;
wherein R₃ is —OH, oxo, —(CH₂)ₙC(O)N(R₄)₂, C₁-C₄ alkyl, or oxadiazolyl, each optionally substituted with one or more R₄ groups;
wherein each R₄ is independently —H or C₁-C₆ alkyl; and
wherein n is 0, 1, or 2,
or a pharmaceutically acceptable salt thereof.

According to another embodiment, R and R′ together form a cyclic ring or a heterocyclic ring containing from 1 to 3 heteroatoms; each optionally substituted with one or more R₃ groups. In some embodiments, R and R′ together form a 3-6 membered monocyclic cycloalkyl or heterocyclic ring containing 1-2 heteroatoms, each optionally substituted with 1-2 R₃ groups.

According to another embodiment, the Formula (I) has the following structure:

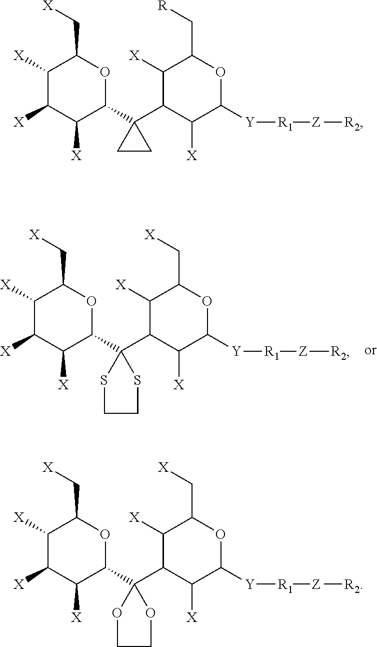

In some embodiments,
Y is —O— or —S—;
Z is absent;
R is ═O, ═NOR₄, or ═C(R₄)₂, and the dashed line representing the second bond is present;
R′ is absent;
R₁ is aryl or a 5-6 membered heterocyclyl or heteroaryl ring containing from 1 to 3 heteroatoms; each optionally substituted with one or more R₃ groups; and
R₂ is —H, aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, aryl, or heteroaryl optionally substituted with one or more R₄ groups;
wherein each R₄ is independently —H or C₁-C₆ alkyl; and
wherein n is 0, 1, or 2,
or a pharmaceutically acceptable salt thereof.

According to another embodiment, the Formula (I) has the following structure:

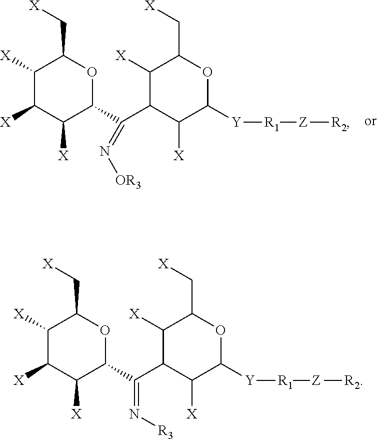

In some embodiments,
Y is —O— or —S—;
Z is absent;
R is —H, —OR₄, halogen, or (C₁-C₆)alkyl, and the dashed line representing the second bond is absent;
R′ is —H, —OR₄, halogen, or (C₁-C₆)alkyl;
R₁ is aryl or a 5-6 membered heterocyclyl or heteroaryl ring containing from 1 to 3 heteroatoms; each optionally substituted with one or more R₃ groups; and
R₂ is —H, or aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, or aryl or heteroaryl optionally substituted with one or more R₄ groups;
wherein each R₄ is independently —H or C₁-C₆ alkyl; and
wherein n is 0, 1, or 2,
or a pharmaceutically acceptable salt thereof.

In other embodiments,
Y is —O— or —S—;
Z is absent;
R is —H, —OR₄, halogen, or (C₁-C₆)alkyl, and the dashed line representing the second bond is absent;
R′ is —H, —OR₄, halogen, or (C₁-C₆)alkyl;
R₁ is aryl optionally substituted with one or more R₃ groups; and
R₂ is —H, or aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, or aryl or heteroaryl optionally substituted with one or more R₄ groups;
wherein each R₄ is independently —H or C₁-C₆ alkyl; and
wherein n is 0, 1, or 2.

According to another aspect of the invention, R₁ is phenyl. In some embodiments, R₁ is phenyl substituted with one or more C₁-C₆ alkyl groups. In yet other embodiments, R₁ is phenyl substituted with one or more methyl groups. In other embodiments, R₁ is C₁-C₆ alkyl. In some embodiments, R₁ is methyl.

According to another aspect of the invention, R₂ is phenyl substituted with one or more R₃ groups. In some embodiments, R₃ is —OH or —(CH₂)ₙC(O)NHR₄. In other embodiments, R₃ is —OH, —CH₂C(O)NHCH₃, or —C(O)NHCH₃.

According to another aspect, R₂ is a 5-6 membered heteroaryl ring containing from 1 to 3 nitrogen atoms; wherein the heteroaryl is optionally substituted with one or more R₃ groups. In some embodiments, R₂ is a diazole optionally substituted with one or more R₃ groups. In other embodiments, R₂ is a diazole optionally substituted with one or more C₁-C₆ alkyl groups. In yet other embodiments, R₂ is a diazole substituted with one or more methyl groups, or a pharmaceutically acceptable salt thereof.

In yet other embodiments, R₂ is phenyl substituted with one or more R₃ groups. In some embodiments, R₃ is —OH or —(CH₂)ₙC(O)NHR₄. In other embodiments, R₃ is —OH, —CH₂C(O)NHCH₃, or —C(O)NHCH₃. In some embodiments, R₂ is absent.

In yet other embodiments, R₂ is —H, phenyl, or a 5-6 membered heterocyclyl or heteroaryl ring containing 1 to 2 nitrogen atoms; each optionally substituted with one or more R₃ groups;

According to one aspect, Y is —O— or —S—. In some embodiments, Y is —O—.

According to another aspect, Z is absent.

According to yet another aspect, R is —H, —OR₄, halogen, or (C₁-C₆)alkyl. In some embodiments, R is —H or OH. In some embodiments, the dashed line representing the second bond is absent. According to another embodiment, R′ is —H.

According to another aspect, R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, aryl or heteroaryl. In some embodiments, the aryl is phenyl and the heteroaryl is a 5-6 membered heteroaryl having 1-4 heteroatom selected from oxygen, nitrogen, or sulfur. In some embodiments, said heteroaryl is oxadiazolyl. In another embodiment, R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, or —NHC(O)NHR₄.

In another embodiment, R₃ is —OH, oxo, —(CH₂)ₙC(O)N(R₄)₂, C₁-C₄ alkyl or oxadiazolyl. In some embodiments, R₃ is OH, —CH₂C(O)NHCH₃, —C(O)NHCH₃, or

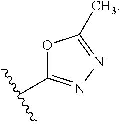 [SMILES: *c1nnc(C)o1]

In some embodiments, each R₃ is optionally substituted with one or more R₄ groups; wherein each R₄ is independently —H or C₁-C₆ alkyl; and wherein n is 0, 1, or 2.

Another embodiment provides a compound represented by a structural formula selected from the group consisting of:

| Cmpd No. | Structure |
|---|---|
| 1 | 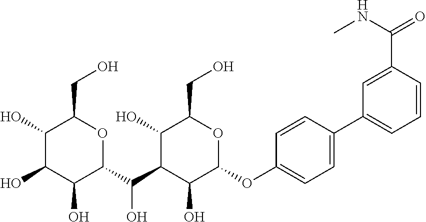 [SMILES: CNC(=O)c1cccc(-c2ccc(O[C@H]3O[C@H](CO)[C@@H](O)[C@H](C(O)[C@H]4O[C@H](CO)[C@@H](O)[C@H](O)[C@@H]4O)[C@@H]3O)cc2)c1] |
| 2 | 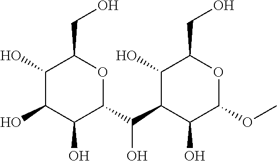 [SMILES: CO[C@H]1O[C@H](CO)[C@@H](O)[C@H](C(O)[C@H]2O[C@H](CO)[C@@H](O)[C@H](O)[C@@H]2O)[C@@H]1O] |
| 3 | 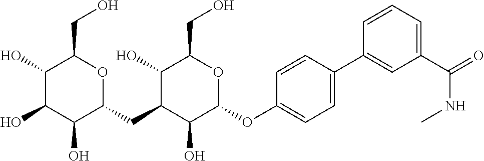 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 4 | 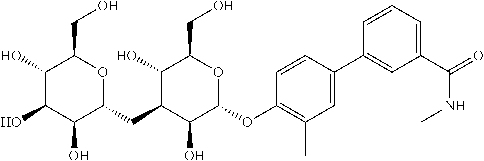 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)c(C)c2)c1] |
| 5 | 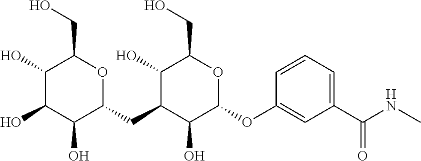 [SMILES: CNC(=O)c1cccc(O[C@H]2O[C@H](CO)[C@@H](O)[C@H](C[C@H]3O[C@H](CO)[C@@H](O)[C@H](O)[C@@H]3O)[C@@H]2O)c1] |
| 6 | 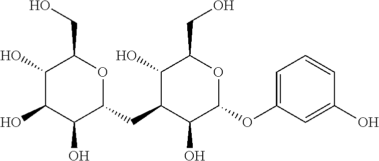 [SMILES: OC[C@H]1O[C@H](C[C@@H]2[C@H](O)[C@@H](Oc3cccc(O)c3)O[C@H](CO)[C@H]2O)[C@@H](O)[C@@H](O)[C@@H]1O] |
| 7 | 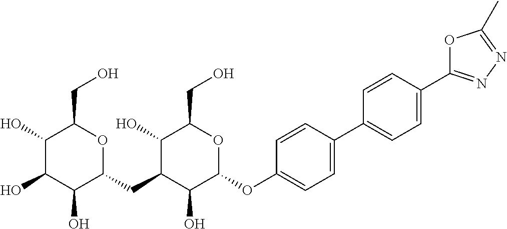 [SMILES: Cc1nnc(-c2ccc(-c3ccc(O[C@H]4O[C@H](CO)[C@@H](O)[C@H](C[C@H]5O[C@H](CO)[C@@H](O)[C@H](O)[C@@H]5O)[C@@H]4O)cc3)cc2)o1] |
| 8 | 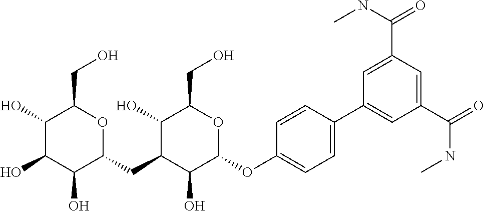 [SMILES: CNC(=O)c1cc(C(=O)NC)cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 9 | 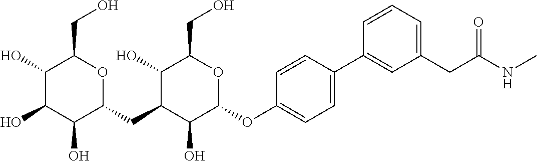 [SMILES: CNC(=O)Cc1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 10 | 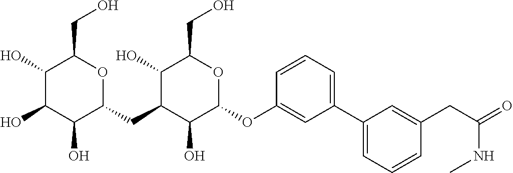 [SMILES: CNC(=O)Cc1cccc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] |
| 11 | 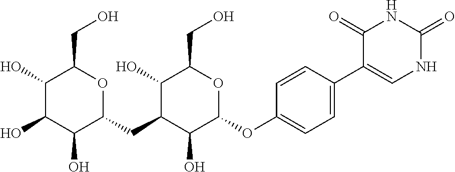 [SMILES: O=c1[nH]cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c(=O)[nH]1] |
| 12 | 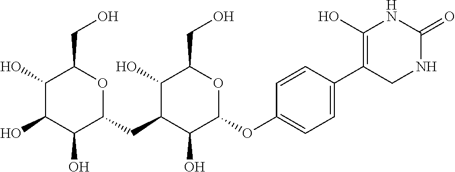 [SMILES: O=C1NCC(c2ccc(O[C@H]3O[C@H](CO)[C@@H](O)[C@H](C[C@H]4O[C@H](CO)[C@@H](O)[C@H](O)[C@@H]4O)[C@@H]3O)cc2)=C(O)N1] |
| 13 | 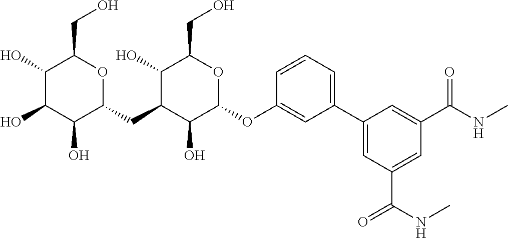 [SMILES: CNC(=O)c1cc(C(=O)NC)cc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] |
| 14 | 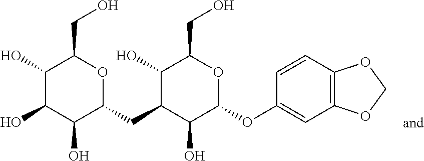 [SMILES: OC[C@H]1O[C@H](C[C@@H]2[C@H](O)[C@@H](Oc3ccc4c(c3)OCO4)O[C@H](CO)[C@H]2O)[C@@H](O)[C@@H](O)[C@@H]1O] |
| 15 | 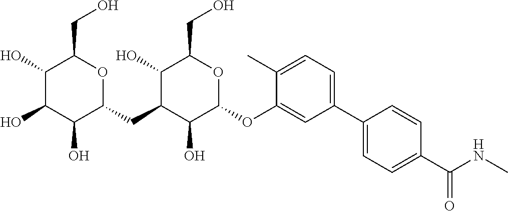 [SMILES: CNC(=O)c1ccc(-c2ccc(C)c(O[C@H]3O[C@H](CO)[C@@H](O)[C@H](C[C@H]4O[C@H](CO)[C@@H](O)[C@H](O)[C@@H]4O)[C@@H]3O)c2)cc1] |

or a pharmaceutically acceptable salt thereof.

The present invention also provides a composition comprising the compound described herein, and a pharmaceutically acceptable carrier, adjuvant, or vehicle.

The present invention also provides a method of treating or preventing bacteria infection in a subject, comprising administering to the subject an effective amount of the compound or the composition described herein.

In an embodiment of the method, the bacteria infection is urinary tract infection or inflammatory bowel disease. In some embodiments, the bacteria infection is a urinary tract infection. In other embodiments, the bacterial infection is involved in inflammatory bowel disease.

Another embodiment provides a method of treating inflammatory bowel disease, such as Crohn's disease or ulcerative colitis, comprising administering to the subject an effective amount of a compound or the composition described herein.

Another embodiment provides method of inhibiting FimH in a cell by contacting the cell with an effective amount a compound or the composition described herein.

Another embodiment provides method of inhibiting adhesion or intracellular replication of AIEC in an epithelial cell by contacting the cell with an effective amount of a compound or the composition described herein.

Another embodiment provides method of blocking the interaction between type 1 pili and CEACAM6 in a cell by contacting the cell with an effective amount of a compound or the composition described herein.

As described herein, a specified number range of atoms includes any integer therein. For example, a group having from 1-4 atoms could have 1, 2, 3, or 4 atoms. The term “stable”, as used herein, refers to compounds that are not substantially altered when subjected to conditions to allow for their production, detection, recovery, storage, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched), or branched, hydrocarbon chain that is completely saturated or that contains one or more units of unsaturation but is non-aromatic. Aliphatic groups include alkyl, alkenyl, and alkynyl groups.

Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds.

The term “cycloaliphatic” (or “carbocycle” or “carbocyclyl” or “carbocyclic”) refers to a non-aromatic monocyclic carbon containing ring which can be saturated or contain one or more units of unsaturation, having three to fourteen ring carbon atoms. The term includes polycyclic fused, spiro or bridged carbocyclic ring systems. The term also includes polycyclic ring systems in which the carbocyclic ring can be fused to one or more non-aromatic carbocyclic or heterocyclic rings or one or more aromatic rings or combination thereof, wherein the radical or point of attachment is on the carbocyclic ring. Fused bicyclic ring systems comprise two rings which share two adjoining ring atoms, bridged bicyclic group comprise two rings which share three or four adjacent ring atoms, spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl. The term “heterocycle” (or “heterocyclyl”, or “heterocyclic”) as used herein means refers to a non-aromatic monocyclic ring which can be saturated or contain one or more units of unsaturation, having three to fourteen ring atoms in which one or more ring carbons is replaced by a heteroatom such as, N, S, or O. The term includes polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, thienothienyl, thienothiazolyl, benzothiolanyl, benzodithianyl, 3-(1-alkyl)-benzimidazol-2-onyl, and 1,3-dihydro-imidazol-2-onyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “alkoxy”, or “thioalkyl”, as used herein, refers to an alkyl group, as previously defined, attached to the molecule through an oxygen (“alkoxy” e.g., —O-alkyl) or sulfur (“thioalkyl” e.g., —S-alkyl) atom.

The terms “haloalkyl”, “haloalkenyl”, “haloaliphatic”, and “haloalkoxy” mean alkyl, alkenyl or alkoxy, as the case may be, substituted with one or more halogen atoms. This term includes perfluorinated alkyl groups, such as —CF₃ and —CF₂CF₃.

The terms “halogen”, “halo”, and “hal” mean F, Cl, Br, or I.

The term “aryl” used alone or as part of a larger moiety as in “aralkyl”, “aralkoxy”, or “aryloxyalkyl”, refers to carbocyclic aromatic ring systems. The term “aryl” may be used interchangeably with the term “aryl ring”.

Carbocyclic aromatic ring groups have only carbon ring atoms (typically six to fourteen, sometimes six to ten) and include monocyclic aromatic rings such as phenyl (C₆aryl), naphthyl (C₁₀aryl), and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring”, as it is used herein, is a group in which an aromatic ring is fused to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The term “heteroaryl”, “heteroaromatic”, “heteroaryl ring”, “heteroaryl group” and “heteroaromatic group”, used alone or as part of a larger moiety as in “heteroaralkyl” or “heteroarylalkoxy”, refers to heteroaromatic ring groups having five to fourteen members, including monocyclic heteroaromatic rings and polycyclic aromatic rings in which a monocyclic aromatic ring is fused to one or more other aromatic ring. Heteroaryl groups have one or more ring heteroatoms. Also included within the scope of the term “heteroaryl”, as it is used herein, is a group in which an aromatic ring is fused to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “protecting group” and “protective group” as used herein, are interchangeable and refer to an agent used to temporarily block one or more desired functional groups in a compound with multiple reactive sites. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

In some embodiments, where indicated a methylene unit of an aliphatic chain is optionally replaced with another atom or group. Examples of such atoms or groups include, but are not limited to, —NR—, —O—, —C(O)—, —C(═N—CN)—, —C(═NR)—, —C(═NOR)—, —S—, —S(O)—, and —S(O)₂—. These atoms or groups can be combined to form larger groups. Examples of such larger groups include, but are not limited to, —OC(O)—, —C(O)CO—, —CO₂—, —C(O)NR—, —C(═N—CN), —NRC(O)—, —NRC(O)O—, —S(O)₂NR—, —NRSO₂—, —NRC(O)NR—, —OC(O)NR—, and —NRSO₂NR—, wherein R is defined herein.

Only those replacement and combinations of groups that result in a stable structure are contemplated. Optional replacements can occur both within the chain and/or at either end of the chain; i.e. both at the point of attachment and/or also at the terminal end. Two optional replacements can also be adjacent to each other within a chain so long as it results in a chemically stable compound. The optional replacements can also completely replace all of the carbon atoms in a chain. For example, a C₃ aliphatic can be optionally replaced by —NR—, —C(O)—, and —NR— to form —NRC(O)NR— (a urea).

Unless otherwise indicated, if the replacement occurs at the terminal end, the replacement atom is bound to an H on the terminal end. For example, if —CH₂CH₂CH₃ were optionally replaced with —O—, the resulting compound could be —OCH₂CH₃, —CH₂OCH₃, or —CH₂CH₂OH.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. For example, a substituent drawn as

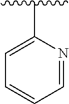 [SMILES: *c1ccccn1]

also represents

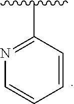 [SMILES: *c1ccccn1]

Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As described herein, where indicated compounds of the invention may optionally be substituted with one or more substituents, such as are illustrated generally herein, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position.

Only those choices and combinations of substituents that result in a stable structure are contemplated. Such choices and combinations will be apparent to those of ordinary skill in the art and may be determined without undue experimentation.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

An aryl group as defined herein may contain one or more substitutable ring atoms, which may be bonded to a suitable substituent. Examples of suitable substituents on a substitutable ring carbon atom of an aryl group include R′. R′ is —Ra, —Br, —Cl, —I, —F, —ORa, —SRa, —O—CORa, —CORa, —CSRa, —CN, —NO₂, —NCS, —SO₃H, —N(RaRb), —COORa, —NRcNRcCORa, —NRcNRcCO₂Ra, —CHO, —CON(RaRb), —OC(O)N(RaRb), —CSN(RaRb), —NRcCORa, —NRcCOORa, —NRcCSRa, —NRcCON(RaRb), —NRcNRcC(O)N(RaRb), —NRcCSN(RaRb), —C(═NRc)—N(RaRb), —C(═S)N(RaRb), —NRd-C(═NRc)—N(RaRb), —NRcNRaRb, —S(O)ₚNRaRb, —NRcSO₂N(RaRb), —NRcS(O)ₚRa, —S(O)ₚRa, —OS(O)ₚNRaRb or —OS(O)ₚRa; wherein p is 1 or 2.

Ra—Rd are each independently —H, an aliphatic group, aromatic group, non-aromatic carbocyclic or heterocyclic group or —N(RaRb), taken together, form a non-aromatic heterocyclic group. The aliphatic, aromatic and non-aromatic heterocyclic group represented by Ra—Rd and the non-aromatic heterocyclic group represented by —N(RaRb) are each optionally and independently substituted with one or more groups represented by R^{#}. Preferably Ra—Rd are unsubstituted.

R^{#} is halogen, R⁺, —OR⁺, —SR⁺, —NO₂, —CN, —N(R⁺)₂, —COR⁺, —COOR⁺, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R)₂, —NHSO₂R⁺, —C(═S)N(R⁺)₂, or —C(═NH)—N(R⁺)₂.

R⁺ is —H, a C₁-C₄ alkyl group, a monocyclic aryl group, a non-aromatic carbocyclic or heterocyclic group each optionally substituted with alkyl, haloalkyl, alkoxy, haloalkoxy, halo, —CN, —NO₂, amine, alkylamine or dialkylamine Preferably R+ is unsubstituted.

An aliphatic or a non-aromatic heterocyclic or carbocyclic group as used herein may contain one or more substituents. Examples of suitable substituents for an aliphatic group or a ring carbon of a non-aromatic heterocyclic group is R″. R″ include those substituents listed above for R′ and ═O, ═S, ═NNHR**, ═NN(R**)₂, ═NNHC(O)R**, ═NNHCO₂ (alkyl), ═NNHSO₂ (alkyl), ═NR**, spiro cycloalkyl group or fused cycloalkyl group. Each R** is independently selected from hydrogen, an unsubstituted alkyl group or a substituted alkyl group. Examples of substituents on the alkyl group represented by R** include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

A preferred position for substitution of a non-aromatic nitrogen-containing heterocyclic group is the nitrogen ring atom. Suitable substituents on the nitrogen of a non-aromatic heterocyclic group or heteroaryl group include —R̂, —N(R̂)₂, C(O)R̂, CO₂R̂, —C(O)C(O)R̂, —SO₂Ŵ, SO₂(R̂)₂, C(═S)N(R̂)₂, C(═NH)—N(R̂)₂, and —NR̂SO₂R̂; wherein R̂ is hydrogen, an aliphatic group, a substituted aliphatic group, aryl, substituted aryl, heterocyclic or carbocyclic ring or a substituted heterocyclic or carbocyclic ring. Examples of substituents on the group represented by R̂ include alkyl, haloalkoxy, haloalkyl, alkoxyalkyl, sulfonyl, alkylsulfonyl, halogen, nitro, cyano, hydroxy, aryl, carbocyclic or heterocyclic ring, oxo, amino, alkylamino, dialkylamino, aminocarbonyl, alkylaminocarbonyl, dialkylaminocarbonyloxy, alkoxy, carboxy, alkoxycarbonyl, or alkylcarbonyl. Preferably R̂ is not substituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

As used herein an optionally substituted aralkyl can be substituted on both the alkyl and the aryl portion. Unless otherwise indicated as used herein optionally substituted aralkyl is optionally substituted on the aryl portion.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds of this invention can exist in free form for treatment, or where appropriate, as a pharmaceutically acceptable salt.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds of this invention, pharmaceutically acceptable derivatives or prodrugs of the compounds of this invention may also be employed in compositions to treat or prevent the herein identified disorders.

As used herein and unless otherwise indicated, the term “prodrug” means a derivative of a compound that can hydrolyze, oxidize, or otherwise react under biological conditions (in vitro or in vivo) to provide a compound of this invention. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of the invention that comprise —NO, —NO₂, —ONO, or —ONO₂ moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

A “pharmaceutically acceptable derivative or prodrug” includes any pharmaceutically acceptable ester, salt of an ester or other derivative or salt thereof of a compound, of this invention which, upon administration to a recipient, is capable of providing, either directly or indirectly, a compound of this invention or an inhibitorily active metabolite or residue thereof. Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

In one embodiment the present invention is a pharmaceutical composition comprising a compound of the present invention and a pharmaceutically acceptable carrier, diluent, adjuvant or vehicle. In one embodiment the present invention is a pharmaceutical composition comprising an effective amount of compound of the present invention and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds of present invention or pharmaceutical salts thereof may be formulated into pharmaceutical compositions for administration to a subject as defined herein. These pharmaceutical compositions, which comprise an amount of the compounds effective to treat or prevent a bacteria infection, such as IBD, and a pharmaceutically acceptable carrier, are another embodiment of the present invention.

In one embodiment the present invention is a method of treating or preventing a bacteria infection, such as IBD, in a subject in need thereof, comprising administering to the subject an effective amount of a compound or composition of the present invention.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, an “effective amount” refers to an amount sufficient to elicit the desired biological response. In the present invention the desired biological response is to reduce or ameliorate the severity, duration, progression, or onset of a bacteria infection, prevent the advancement of a bacteria infection, cause the regression of a bacteria infection, prevent the recurrence, development, onset or progression of a symptom associated with a bacteria infection, or enhance or improve the prophylactic or therapeutic effect(s) of another therapy. The precise amount of compound administered to a subject will depend on the mode of administration, the type and severity of the disease or condition and on the characteristics of the subject, such as general health, age, sex, body weight and tolerance to drugs. It will also depend on the degree, severity and type of bacteria infection, and the mode of administration. The skilled artisan will be able to determine appropriate dosages depending on these and other factors. When co-administered with other agents, e.g., when co-administered with a bacteria infection agent, an “effective amount” of the second agent will depend on the type of drug used. Suitable dosages are known for approved agents and can be adjusted by the skilled artisan according to the condition of the subject, the type of condition(s) being treated and the amount of a compound of the invention being used. In cases where no amount is expressly noted, an effective amount should be assumed.

As used herein, the terms “treat”, “treatment” and “treating” refer to the reduction or amelioration of the progression, severity and/or duration of a bacteria infection, or the amelioration of one or more symptoms (preferably, one or more discernible symptoms) of a bacteria infection resulting from the administration of one or more therapies (e.g., one or more therapeutic agents such as a compound of the invention). In specific embodiments, the terms “treat”, “treatment” and “treating” refer to the amelioration of at least one measurable physical parameter of a bacteria infection. In other embodiments the terms “treat”, “treatment” and “treating” refer to the inhibition of the progression of a bacteria infection, either physically by, e.g., stabilization of a discernible symptom, physiologically by, e.g., stabilization of a physical parameter, or both. In other embodiments the terms “treat”, “treatment” and “treating” refer to the reduction or stabilization of a bacteria infection.

As used herein, the terms “prevent”, “prevention” and “preventing” refer to the reduction in the risk of acquiring or developing a given bacteria infection, or the reduction or inhibition of the recurrence or a bacteria infection. In one embodiment, a compound of the invention is administered as a preventative measure to a patient, preferably a human, having a genetic predisposition to any of the conditions, diseases or disorders described herein.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like. The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions of the present invention may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The dosage regimen utilizing the compounds of present invention can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compound of present invention required to treat, for example, to prevent, inhibit (fully or partially) or arrest the progress of the disease.

Dosages of the compounds of present invention can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosings such as twice, three or four times per day.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

An effective amount can be achieved in the method or pharmaceutical composition of the invention employing a compound of present invention or a pharmaceutically acceptable salt thereof alone or in combination with an additional suitable therapeutic agent, for example, a cancer-therapeutic agent. When combination therapy is employed, an effective amount can be achieved using a first amount of a compound of present invention or a pharmaceutically acceptable salt thereof and a second amount of an additional suitable therapeutic agent.

In one embodiment, the compound of present invention and the additional therapeutic agent, are each administered in an effective amount (i.e., each in an amount which would be therapeutically effective if administered alone). In another embodiment, the compound of present invention and the additional therapeutic agent, are each administered in an amount which alone does not provide a therapeutic effect (a sub-therapeutic dose). In yet another embodiment, the compound of present invention can be administered in an effective amount, while the additional therapeutic agent is administered in a sub-therapeutic dose. In still another embodiment, the compound of present invention can be administered in a sub-therapeutic dose, while the additional therapeutic agent, for example, a suitable cancer-therapeutic agent is administered in an effective amount.

As used herein, the terms “in combination” or “coadministration” can be used interchangeably to refer to the use of more than one therapies (e.g., one or more prophylactic and/or therapeutic agents). The use of the terms does not restrict the order in which therapies (e.g., prophylactic and/or therapeutic agents) are administered to a subject. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

When coadministration involves the separate administration of the first amount of a compound of present invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of present invention and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

It is understood that the method of coadministration of a first amount of a compound of present invention and a second amount of an additional therapeutic agent can result in an enhanced or synergistic therapeutic effect, wherein the combined effect is greater than the additive effect that would result from separate administration of the first amount of the compound of present invention and the second amount of the additional therapeutic agent.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

The activity of the compounds as inhibitors of bacteria infection may be assayed in vitro or in vivo. In vitro assays include assays that determine inhibition of the FimH activity. Alternate in vitro assays quantitate the ability of the inhibitor to bind to the FimH and may be measured either by radiolabelling the inhibitor prior to binding, isolating the inhibitor complex and determining the amount of radiolabel bound, or by running a competition experiment where new inhibitors are incubated with the FimH bound to known radioligands. Detailed conditions for assaying a compound utilized in this invention are set forth in the Examples below.

### EXPERIMENTAL DETAILS

The following abbreviations are used in the examples below:

AcOH acetic acid
AC₂O acetic anhydride
AIBN azobisisobutyronitrile
aq aqueous
BF₃.OEt₂ diethyloxonio-trifluoro-boron
CH₃CN acetonitrile
CDCl₃ chloroform-D
conc concentrate
CV column volume
Cs₂CO₃ cesium carbonate
Cu(OAc)₂ diacetoxy copper
DBU 1,8-diazabicyclo[5.4.0]undec-7-ene
DCM methylene chloride or dichloromethane
DMAP 4-dimethylaminopyridine
DMF dimethylformamide
DMSO dimethylsulfoxide
Eq. equivalent
EtOAc ethyl acetate
h hour
Hex hexanes

### IPA Isopropanol

LiOH.H₂O lithium hydroxide monohydrate
M molar
MeOH methanol
NaOMe sodium methoxide
Min minute
MS 4 Å molecular sieves 4 angstrom
MTBE methyl tert-butyl ether
Na₂SO₄ sodium sulfate

### NMO N-methylmorpholine-N-oxide

OsO₄ osmium tetroxide
PdCl₂ palladium (II)chloride
Pd(OAc)₂ palladium (II)acetate
PdCl₂(dppf)₂.DCM (1,1′-Bis-(diphenylphosphino)-ferrocene)palladium (II) dichloride
Pd(OH)₂ dihydroxy palladium
Pd(PPh₃)₄ tetrakis(triphenylphosphine) palladium
Py pyridine
rt room temperature
Rt retention time
TEA triethylamine
THF tetrahydrofuran
TLC thin layer chromatography
TMSOTf trimethylsilyl trifluoromethanesulfonate

The following is a list of key intermediates which are used in the preparation of compounds in the examples.

### Preparation of Intermediate I ((1R,2S,3S,4S,5R)-2,4-Dibenzyloxy-3-vinyl-6,8-dioxabicyclo[3.2.1]octane)

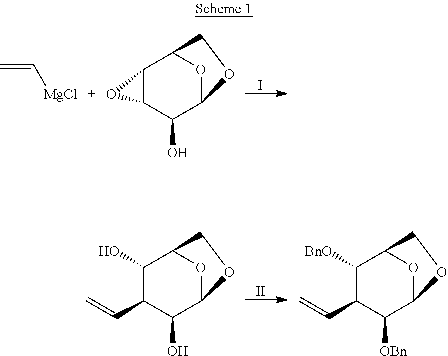

### Step I. (1R,2S,3S,4S,5R)-3-Vinyl-6,8-dioxabicyclo[3.2.1]octane-2,4-diol

To a solution of (1R,2R,4S,5S,6R)-3,7,9-trioxatricyclo[4.2.1.2,4]nonan-5-ol (1.5 g, 10.41 mmol) in THF (20 mL) is added chloro-vinyl-magnesium (19.52 mL of 1.6 M, 31.23 mmol) dropwise and the mixture is stirred at 60° C. for 4 h under nitrogen. After quenching with 0.5 mL of sat. NH₄Cl solution, the mixture is diluted with DCM/MeOH (95/5), filtered on a pad of celite, washed with DCM/MeOH (9/1). The filtrate is concentrated, and then the residue is dissolved in 10% MeOH/DCM, co-evaporated with silica, dried and purified on a short silica gel column using 5 to 10% MeOH in DCM to obtain the title compound (1.8 g) as a yellow oil, which solidifies upon standing.

### Step II: Intermediate I (1R,2S,3S,4S,5R)-2,4-Dibenzyloxy-3-vinyl-6,8-dioxabicyclo[3.2.1]octane

To a solution of (1R,2S,3S,4S,5R)-3-vinyl-6,8-dioxabicyclo[3.2.1]octane-2,4-diol (1.8 g, 10.45 mmol) in DMF (20 mL) is added 60% NaH (1.249 g, 31.23 mmol) by portions under N₂ at 0° C. After stirring for 20 min at 0° C., benzyl bromide (2.7 mL, 22.70 mmol) is added followed by a cat. TBAI (192.3 mg, 0.5205 mmol). The mixture is stirred at rt for 5 h. It is then diluted with ether, quenched by addition of 10 drops of methanol, washed with water and brine consecutively, dried over sodium sulfate, concentrated to dryness. The residue is separated on Biotage™ SNAP 100 g silica gel cartridge using a gradient of ethyl acetate/hexanes 0-20% in 20 CV to afford the title compound (2.82 g) as an oil.

1H NMR (400 MHz, CDCl₃) δ 7.36-7.24 (m, 10H), 6.32 (m, 1H), 5.47 (d, 1H), 5.15 (m, 2H), 4.63 (m, 3H), 4.57 (m, 1H), 4.39 (d, 1H), 3.84 (m, 1H), 3.73 (m, 2H), 3.43 (m, 1H), 3.12 (m, 1H).

### Preparation of Intermediate II ((1R,2S,3S,4S,5R)-2,4-Dibenzyloxy-6,8-dioxabicyclo[3.2.1]octane-3-carbaldehyde)

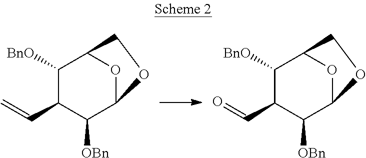 [SMILES: C=C[C@@H]1[C@H](OCc2ccccc2)[C@@H]2OC[C@@H](O2)[C@H]1OCc1ccccc1 ; O=C[C@@H]1[C@H](OCc2ccccc2)[C@@H]2OC[C@@H](O2)[C@H]1OCc1ccccc1]

Intermediate I (2.72 g, 7.718 mmol) in DCM (28 mL)/MeOH (12 mL) is kept for ozonolysis at −78° C. After a sustainable blue color is obtained, the excess ozone is flushed out with nitrogen for 7 min at the same temperature and then triphenylphosphine (4.454 g, 16.98 mmol) is added. The mixture is warmed up to rt in 20 min and stirred for another 30 min. After removal of the solvent under reduced pressure, the residue is purified on Biotage™ SNAP 100 g silica gel cartridge using a gradient of ethyl acetate/hexanes 0-25% in 20 CV to obtain the title compound (2.2 g) as an oil.

1H NMR (400 MHz, CDCl₃) δ 9.99 (s, 1H), 7.40-7.26 (m, 10H), 5.50 (s, 1H), 4.73 (d, 1H), 4.64 (d, 1H), 4.55 (s, 2H), 4.44 (d, 1H), 4.19-4.03 (m, 2H), 3.67-3.49 (m, 2H), 2.82 (d, 1H).

### Preparation of Intermediate III (2-[(2R,3S,4S,5R,6R)-3,4,5-Tribenzyloxy-6-(benzyloxymethyl)tetrahydropyran-2-yl]sulfonylpyridine)

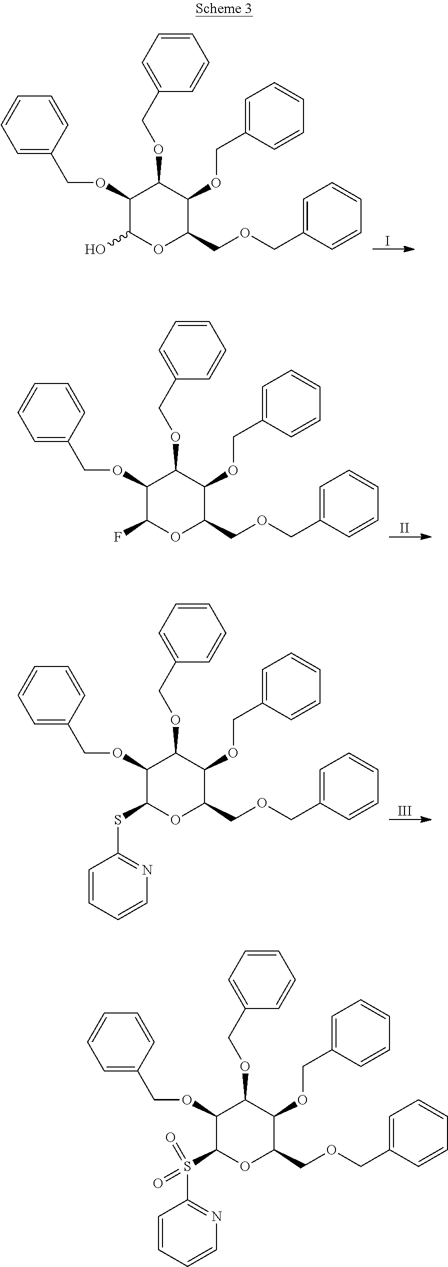

### Step I. (2R,3R,4S,5S,6R)-3,4,5-Tribenzyloxy-2-(benzyloxymethyl)-6-fluoro-tetrahydropyran

To a solution of (3S,4S,5R,6R)-3,4,5-tribenzyloxy-6-(benzyloxymethyl)tetrahydropyran-2-ol (10.8 g, 19.98 mmol) and (diethylamino)difluorosulfonium tetrafluoroborate (7.075 g, 29.97 mmol) in 50 mL of DCM is added DBU (4.8 mL, 32.10 mmol) at −15° C. and then stirred for 20 min. The reaction is quenched by adding saturated sodium bicarbonate solution. Then the mixture is extracted with DCM (3×20 mL). The combined organic extracts are washed with water and brine consecutively, dried over sodium sulfate, filtered, and concentrated to dryness. The residue is separated on Biotage™ SNAP 100 g silica gel cartridge using a gradient of ethyl acetate/hexanes 0-15% in 20 CV to obtain a major fraction containing the title compound (6.4 g).

LC-MS: m/z=565.4 (M+Na⁺)

### Step II. 2-[(2R,3S,4S,5R,6R)-3,4,5-Tribenzyloxy-6-(benzyloxymethyl)tetrahydropyran-2-yl]sulfanylpyridine

To a solution of (2R,3R,4S,5S,6R)-3,4,5-tribenzyloxy-2-(benzyloxymethyl)-6-fluoro-tetrahydropyran (2000 mg, 3.686 mmol), pyridine-2-thiol (532.7 mg, 4.792 mmol) and 4 A MS (1500 mg) in DCM (30 mL) is added BF₃ etherate (591.4 μL, 4.792 mmol) at 0° C. and the mixture is then stirred at rt for 20 min. It is then diluted with DCM (20 mL), quenched with sat. NaHCO₃ solution (10 mL), washed with water and brine consecutively, dried over sodium sulfate, and concentrated to dryness. The residue is separated on Biotage™ SNAP 100 g silica gel cartridge using a gradient of ethyl acetate/hexanes 0-25% in 20 CV to obtain the title compound (1.54 g) as a major product.

### Step III: Intermediate III (2-[(2R,3S,4S,5R,6R)-3,4,5-Tribenzyloxy-6-(benzyloxymethyl)tetrahydropyran-2-yl]sulfonylpyridine)

To a solution of 2-[(2R,3S,4S,5R,6R)-3,4,5-tribenzyloxy-6-(benzyloxymethyl)tetrahydropyran-2-yl]sulfanylpyridine (3.9 g, 6.153 mmol) in DCM (25 mL) is added MCPBA (4.137 g, 18.46 mmol) at 0° C. and the mixture is then stirred at rt for 1 h. It is then diluted with DCM (30 mL), quenched with sat. NaHSO₃ solution and sat. sodium bicarbonate. The aqueous phase is separated from the organic phase using a separatory funnel, extracted with DCM (2×20 mL). The combined organic phases are washed with water and brine consecutively, dried over sodium sulfate, and concentrated to dryness. The residue is separated on Biotage™ SNAP 100 g silica gel cartridge using a gradient of ethyl acetate/hexanes 0-30% in 20 CV to obtain the title compound (3.6 g).

1H NMR (400 MHz, CDCl₃) δ 8.74 (m, 1H), 8.07-7.96 (m, 1H), 7.68 (m, 1H), 7.45 (m, 1H), 7.39 (m, 2H), 7.34-7.22 (m, 14H), 7.21-7.12 (m, 4H), 5.51 (d, 1H), 4.82 (d, 1H), 4.75 (d, 1H), 4.69-4.63 (m, 2H), 4.60 (d, 2H), 4.54-4.38 (m, 3H), 4.32-4.20 (m, 2H), 3.97-3.89 (m, 1H), 3.59-3.50 (m, 2H).

### Preparation of Intermediate IV ([(2R,3R,4R,5R,6R)-6-[[(2R,3S,4S,5S,6R)-2-Acetoxy-6-(acetoxymethyl)-3,5-dibenzyloxy-tetrahydropyran-4-yl]methyl]-3,4,5-tribenzyloxy-tetrahydropyran-2-yl]methyl acetate)

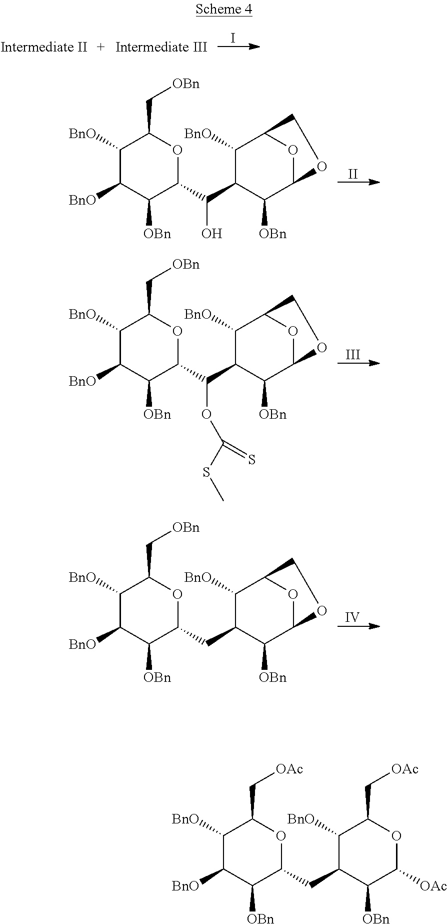

### Step I. [(1R,2S,3S,4S,5R)-2,4-Dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methanol

A stirred solution of Intermediate II (281.9 mg, 0.7955 mmol) and Intermediate III (900 mg, 1.352 mmol) in THF (6 mL) is flushed with nitrogen for 5 min, and then to it is added samarium(II) iodide/THF (30.23 mL of 0.1 M, 3.023 mmol) dropwise till a persistent dark blue color is reached at rt under nitrogen. The mixture is quenched with 0.3 ml of sat. NH₄Cl solution and filtered over a pad of celite. The filtrate is concentrated to dryness. The residue is separated on Biotage™ SNAP 50 g silica gel cartridge using a gradient of ethyl acetate/hexanes 0-25% in 20 CV to obtain an inseparable mixture (550 mg) containing the title compound, which is used directly in the next step without further purification.

LC-MS: m/z=880.7 (M+H⁺)

### Step II. O-[[(1R,2S,3R,4S,5R)-2,4-dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methyl]methylsulfanylmethanethioate

To a solution containing [(1R,2S,3S,4S,5R)-2,4-dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methanol (240 mg, 0.2470 mmol) in THF (3 mL) is added 60% NaH (27.7 mg, 0.6940 mmol) at 0° C. and the mixture is stirred at rt for 30 min under nitrogen. Then, methanedithione (52.84 mg, 41.7 μL, 0.6940 mmol) is added to it. After stirring for 30 min at rt, iodomethane (98.51 mg, 43.2 μL, 0.6940 mmol) is added to the mixture and stirred at rt for 1 h. It is then diluted with ether (20 mL), quenched with a drop of acetic acid, washed with water and brine consecutively, dried over sodium sulfate, and concentrated to dryness. The residue is separated on Biotage™ SNAP 25 g silica gel cartridge using a gradient of ethyl acetate/hexanes 0-25% in 20 CV to obtain a fraction, mainly containing the desired product, which is used directly to the next step without further purification.

LC-MS: m/z=992.7 (M+Na⁺)

### Step III. (1R,2S,3S,4S,5R)-2,4-Dibenzyloxy-3-[[(2R,3R,4R,5R,6R)-3,4,5-tribenzyloxy-6-(benzyloxymethyl)tetrahydropyran-2-yl]methyl]-6,8-dioxabicyclo[3.2.1]octane

To the mixture containing O-[[(1R,2S,3R,4S,5R)-2,4-dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methyl]methylsulfanylmethanethioate (240 mg, 0.2476 mmol) in toluene (3 mL) are added AIBN (16.2 mg, 0.0987 mmol) and tributyltin hydride (199 μL, 0.741 mmol). The mixture is flushed with nitrogen for 10 min and then is heated at reflux for 1 h. After removal of the solvent under reduced pressure, the residue is separated on Biotage™ SNAP 25 g silica gel cartridge using a gradient of ethyl acetate/hexanes 0-25% in 30 CV to obtain a mixture (200 mg), mainly containing the title compound, which is used directly in the next step without further purification.

LC-MS: m/z=863.7 (M+H⁺)

### Step IV: Intermediate IV ([(2R,3R,4R,5R,6R)-6-[[(2R,3S,4S,5S,6R)-2-Acetoxy-6-(acetoxymethyl)-3,5-dibenzyloxy-tetrahydropyran-4-yl]methyl]-3,4,5-tribenzyloxy-tetrahydropyran-2-yl]methyl acetate)

To a stirred solution of (1R,2S,3S,4S,5R)-2,4-dibenzyloxy-3-[[(2R,3R,4R,5R,6R)-3,4,5-tribenzyloxy-6-(benzyloxymethyl)tetrahydropyran-2-yl]methyl]-6,8-dioxabicyclo[3.2.1]octane (660 mg, 0.7647 mmol) in Ac₂O (1 mL, 10.60 mmol) is added TFA (100 μL, 1.298 mmol), and then stirred at rt. The reaction is carefully monitored with

LC-MS till the starting material disappears (about 2 h). Then the volatiles are removed under reduced pressure. The residue is separated on Biotage™ SNAP 25 g silica gel cartridge using a gradient of ethyl acetate/hexanes 0-30% in 20 CV to obtain a mixture (580 mg), mainly containing the title compound, which is used directly to the next step without further purification.

LC-MS: m/z=939.5 (M+Na⁺)

### Preparation of Intermediate V ([(2R,3R,4R,5R,6R)-6-[[(2R,3S,4S,5S,6R)-2-(Acetoxymethyl)-3,5-dibenzyloxy-6-(4-bromophenoxy)tetrahydropyran-4-yl]methyl]-3,4,5-tribenzyloxy-tetrahydropyran-2-yl]methyl acetate)

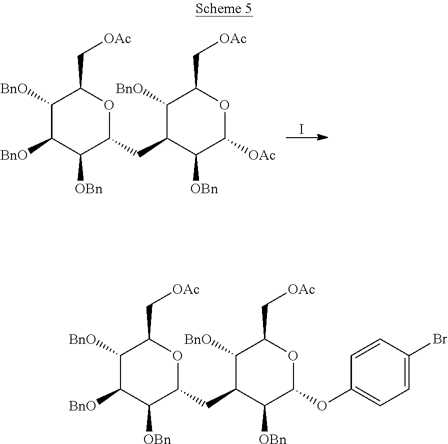

To a stirred solution of Intermediate IV (115 mg, 0.1254 mmol) and 4-bromophenol (43.4 mg, 0.2509 mmol) in DCM (3 mL) is added BF₃ etherate (31 μL, 0.2512 mmol) at 0° C. The mixture is stirred at rt under nitrogen till the starting material disappears (about 2 h). After removal of the solvent under reduced pressure, the residue is separated on Biotage™ SNAP 25 g silica gel cartridge using a gradient of ethyl acetate/hexanes 0-25% in 20 CV to obtain a mixture (101 mg), mainly containing the title compound, which is used directly in the next step without further purification.

LC-MS: m/z=1051.7 (M+Na⁺).

### Preparation of Intermediate VI ([(2R,3R,4R,5R,6R)-6-[[(2R,3S,4S,5S,6R)-2-(Acetoxymethyl)-3,5-dibenzyloxy-6-(3-bromophenoxy)tetrahydropyran-4-yl]methyl]-3,4,5-tribenzyloxy-tetrahydropyran-2-yl]methyl acetate)

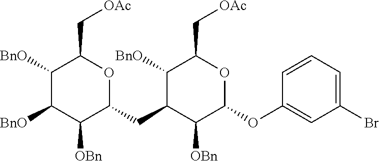 [SMILES: CC(=O)OC[C@H]1O[C@H](Oc2cccc(Br)c2)[C@@H](OCc2ccccc2)[C@@H](C[C@H]2O[C@H](COC(C)=O)[C@@H](OCc3ccccc3)[C@H](OCc3ccccc3)[C@@H]2OCc2ccccc2)[C@@H]1OCc1ccccc1]

The title compound is prepared using a procedure similar to that described for the preparation of Intermediate V in Scheme 5.

LC-MS: m/z=1051.7 (M+Na⁺).

### Example 1

### Preparation of Compound 1 (3-[4-[(2R,3S,4R,5S,6R)-3,5-Dihydroxy-6-(hydroxymethyl)-4-[hydroxy-[(2R,3S,4S,5S,6R)-3,4,5-trihydroxy-6-(hydroxymethyl)tetrahydropyran-2-yl]methyl]tetrahydropyran-2-yl]oxyphenyl]-N-methyl-benzamide)

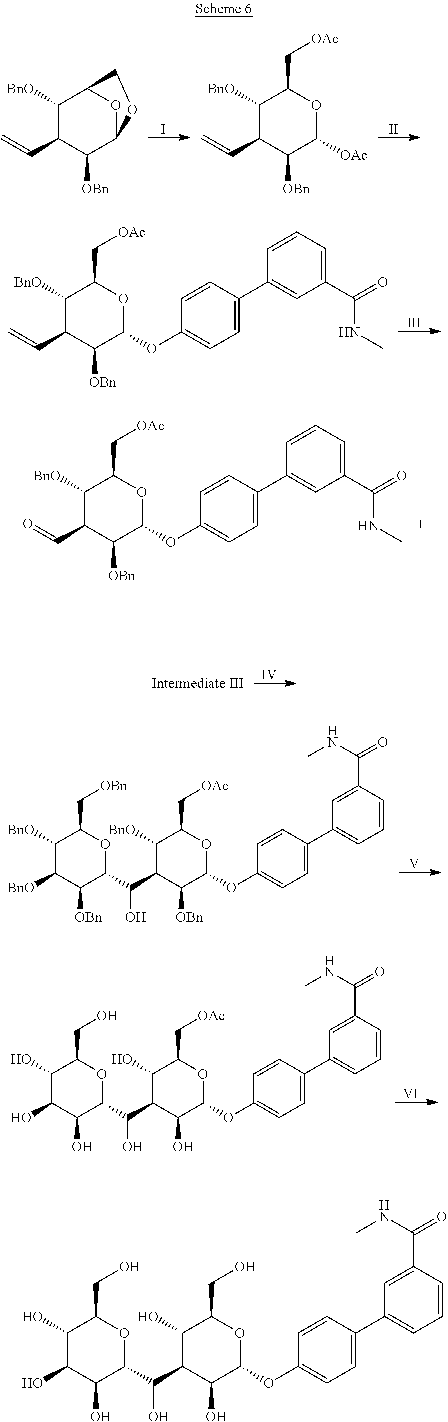

### Step I. [(2R,3S,4S,5S,6R)-6-Acetoxy-3,5-dibenzyloxy-4-vinyl-tetrahydropyran-2-yl]methyl acetate

To a solution of Intermediate I (336 mg, 0.9534 mmol) in AC₂O (7.5 mL) is added TFA (750 μL, 9.735 mmol), and the mixture is stirred at rt overnight. Then it is co-evaporated with toluene and the crude mixture is separated on Biotage™ SNAP 25 g silica gel cartridge using a gradient of EtOAc/hexanes 0-20% in 20 CV to obtain the title compound (280 mg).

LC-MS: m/z=477.4 (M+Na⁺).

### Step II. [(2R,3S,4S,5S,6R)-3,5-Dibenzyloxy-6-[4-[3-(methylcarbamoyl)phenyl]phenoxy]-4-vinyl-tetrahydropyran-2-yl]methyl acetate

To a solution of [(2R,3S,4S,5S,6R)-6-acetoxy-3,5-dibenzyloxy-4-vinyl-tetrahydropyran-2-yl]methyl acetate (160 mg, 0.3520 mmol) and 3-(4-hydroxyphenyl)-N-methyl-benzamide (160.0 mg, 0.7040 mmol) in DCM (3 mL) is added BF₃ etherate (80 μL, 0.7040 mmol) at rt. Then it is heated to 40° C. under nitrogen overnight. After removal of the solvent under reduced pressure, the residue is purified on Biotage™ SNAP 25 g silica gel cartridge using a gradient of ethyl acetate/hexanes 0-60% in 20 CV to obtain the title compound (200 mg).

### Step III. [(2R,3S,4S,5S,6R)-3,5-Dibenzyloxy-4-formyl-6-[4-[3-(methylcarbamoyl)phenyl]phenoxy]tetrahydropyran-2-yl]methyl acetate

[(2R,3S,4S,5S,6R)-3,5-dibenzyloxy-6-[4-[3-(methylcarbamoyl)phenyl]phenoxy]-4-vinyl-tetrahydropyran-2-yl]methyl acetate (200 mg, 0.3217 mmol) in DCM (12 mL)/MeOH (3 mL) is kept for ozonolysis at −78° C. After a sustainable blue color is obtained, the excess nitrogen is flushed out with nitrogen for 7 min at the same temperature and then triphenylphosphine (185.6 mg, 0.7077 mmol) is added to it. The mixture is warmed up to rt in 20 min and stirred for another 30 min. After removal of the solvent under reduced pressure, the residue is purified on Biotage™ SNAP 25 g silica gel cartridge using a gradient of EtOAc/hexanes 0-60% in 20 CV to obtain the title compound (164 mg).

### Step IV. ((2R,3S,4S,5S,6R)-3,5-Bis(benzyloxy)-4-(hydroxy((2R,3S,4S,5R,6R)-3,4,5-tris(benzyloxy)-6-((benzyloxy)methyl)tetrahydro-2H-pyran-2-yl)methyl)-6-((3′-(methylcarbamoyl)-[1,1′-biphenyl]-4-yl)oxy)tetrahydro-2H-pyran-2-yl)methyl acetate

A solution of [(2R,3S,4S,5S,6R)-3,5-dibenzyloxy-4-formyl-6-[4-[3-(methylcarbamoyl)phenyl]phenoxy]tetrahydropyran-2-yl]methyl acetate (52.3 mg, 0.0838 mmol) and Intermediate III (96 mg, 0.1442 mmol) in THF (4 mL) is flushed with nitrogen for 5 min. Then to it is added samarium (II) iodide (4 mL of 0.1 M, 0.4000 mmol) dropwise till a persistent blue color is reached (about 4 mL). The mixture is quenched with a few drops of sat. NH₄Cl solution and filtered over a pad of celite. The filtrate is concentrated to dryness. The residue is separated on Biotage™ SNAP 25 g silica gel cartridge using a gradient of ethyl acetate/hexanes 0-50% in 20 CV to obtain an inseparable mixture (91 mg) containing the title compound. The mixture is used directly in the next step without further purification.

### Step V. [(2R,3S,4R,5S,6R)-3,5-Dihydroxy-4-[hydroxy-[(2R,3S,4S,5S,6R)-3,4,5-trihydroxy-6-(hydroxymethyl)tetrahydropyran-2-yl]methyl]-6-[4-[3-(methylcarbamoyl)phenyl]phenoxy]tetrahydropyran-2-yl]methyl acetate

To a solution of ((2R,3S,4S,5S,6R)-3,5-bis(benzyloxy)-4-(hydroxy((2R,3S,4S,5R,6R)-3,4,5-tris(benzyloxy)-6-((benzyloxy)methyl)tetrahydro-2H-pyran-2-yl)methyl)-6-((3′-(methylcarbamoyl)-[1,1′-biphenyl]-4-yl)oxy)tetrahydro-2H-pyran-2-yl)methyl acetate (50 mg, 0.0435 mmol) in MeOH (3 mL) is added 20% Pd(OH)₂/C (6.1 mg) and 2 drops of acetic acid. Then the mixture is subject to hydrogenation using a H₂ balloon and the mixture is stirred at rt overnight. After filtration, the filtrate is concentrated to dryness and the residue containing the title compound (25 mg) is used directly in the next step.

### Step VI: Compound 1 (3-[4-[(2R,3S,4R,5S,6R)-3,5-Dihydroxy-6-(hydroxymethyl)-4-[hydroxy-[(2R,3S,4S,5S,6R)-3,4,5-trihydroxy-6-(hydroxymethyl)tetrahydropyran-2-yl]methyl]tetrahydropyran-2-yl]oxyphenyl]-N-methyl-benzamide)

To a solution of [(2R,3S,4R,5S,6R)-3,5-Dihydroxy-4-[hydroxy-[(2R,3S,4S,5S,6R)-3,4,5-trihydroxy-6-(hydroxymethyl)tetrahydropyran-2-yl]methyl]-6-[4-[3-(methylcarbamoyl)phenyl]phenoxy]tetrahydropyran-2-yl]methyl acetate (25 mg) in MeOH (2 mL) is added 25% sodium methoxide (20 μL, 0.3482 mmol) and the mixture is stirred at rt overnight. After neutralization with resin Amberlite IR120 (H), the mixture is filtered and the filtrate is concentrated to dryness. The residue is purified using reverse-phase prep-HPLC to provide the title compound (13 mg) as a white solid.

1H NMR (CD₃OD, 400 MHz) δ 8.01 (m, 1H), 7.73 (m, 2H), 7.67-7.54 (m, 2H), 7.49 (m, 1H), 7.22 (d, 2H), 5.40 (d, 1H), 4.48 (m, 1H), 4.44-4.38 (m, 1H), 4.19-3.87 (m, 4H), 3.87-3.62 (m, 7H), 2.92 (s, 3H), 2.23 (m, 1H) LC-MS: m/z=566.4 (M+H⁺).

### Example 2

### Preparation of Compound 2 ((2R,3S,4S,5S,6R)-2-[[(2R,3S,4R,5S,6S)-3,5-Dihydroxy-2-(hydroxymethyl)-6-methoxy-tetrahydropyran-4-yl]-hydroxy-methyl]-6-(hydroxymethyl)tetrahydropyran-3,4,5-triol)

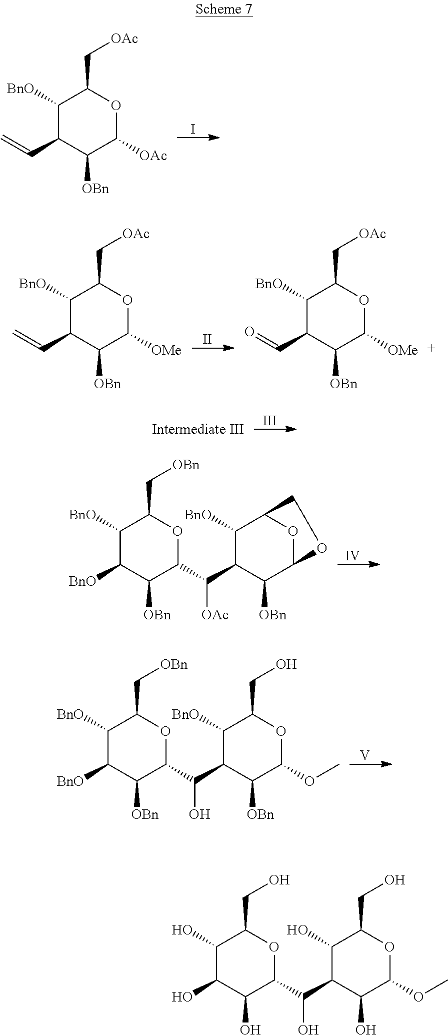

### Step I. [(2R,3S,4S,5S,6S)-3,5-Dibenzyloxy-6-methoxy-4-vinyl-tetrahydropyran-2-yl]methyl acetate

To a solution of [(2R,3S,4S,5S,6R)-6-acetoxy-3,5-dibenzyloxy-4-vinyl-tetrahydropyran-2-yl]methyl acetate (296 mg, 0.6512 mmol) in DCM (3 mL) are added MeOH (250 μL, 6.172 mmol) and BF₃ etherate (184.8 mg, 161 μL, 1.303 mmol). The mixture is heated to reflux under nitrogen overnight. After removal of the solvent, the residue is purified on Biotage™ SNAP 25 g silica gel cartridge using a gradient of ethyl acetate/hexanes 0-25% in 20 CV to obtain the title compound (103 mg).

### Step II. [(2R,3S,4S,5S,6S)-3,5-Dibenzyloxy-4-formyl-6-methoxy-tetrahydropyran-2-yl]methyl acetate

[(2R,3S,4S,5S,6S)-3,5-Dibenzyloxy-6-methoxy-4-vinyl-tetrahydropyran-2-yl]methyl acetate (235 mg) in DCM (12 mL)/MeOH (4 mL) is subject to ozonolysis at −78° C. After a sustainable blue color (about 5 min) is obtained, the excess ozone is flushed out with nitrogen for 7 min at the same temperature and then triphenylphosphine (298.2 mg, 1.137 mmol) is added. The mixture is warmed up to rt in 20 min and stirred for another 30 min. After removal of the solvent under reduced pressure, the residue is purified on Biotage™ SNAP 25 g silica gel cartridge using a gradient of ethyl acetate/hexanes 0-25% in 20 CV to obtain the title compound (214 mg).

### Step III. [[(1R,2R,3R,4S,5R)-2,4-Dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methyl]acetate

A stirred solution of [(2R,3S,4S,5S,6S)-3,5-dibenzyloxy-4-formyl-6-methoxy-tetrahydropyran-2-yl]methyl acetate (132.8 mg, 0.3100 mmol) and Intermediate III (372 mg, 0.5587 mmol) in THF (6 mL) is flushed with nitrogen for 5 min, and then to it is added samarium(II) iodide/THF (12.4 mL of 0.1 M, 1.240 mmol) dropwise till a persistent dark blue color is reached at rt under nitrogen. The mixture is quenched with 0.2 ml of sat. NH₄Cl solution and filtered over a pad of celite. The filtrate is concentrated to dryness. The residue is separated on Biotage™ SNAP 25 g silica gel cartridge using a gradient of ethyl acetate/hexanes 0-25% in 20 CV to obtain an inseparable mixture (260 mg) containing the title compound.

LC-MS: m/z=921.7 (M+H⁺).

### Step IV. [(2R,3S,4S,5S,6S)-3,5-Dibenzyloxy-2-(hydroxymethyl)-6-methoxy-tetrahydropyran-4-yl]-[(2R,3S,4S,5R,6R)-3,4,5-tribenzyloxy-6-(benzyloxymethyl)tetrahydropyran-2-yl]methanol

To a mixture containing [[(1R,2R,3R,4S,5R)-2,4-dibenzyloxy-6,8-dioxabicyclo[3.2.1]octan-3-yl]-[(2R,3S,4S,5R,6R)-3,4,5-tribenzyloxy-6-(benzyloxymethyl)tetrahydropyran-2-yl]methyl]acetate (260 mg) in MeOH (5 mL) is added 25% sodium methoxide (17 μL, 0.3102 mmol) and the mixture is stirred at rt overnight. It is then neutralized with resin Amberlite IR120 (H). After filtration, the solvent is removed under reduced pressure to obtain a mixture (143 mg), containing the title compound.

1H NMR (400 MHz, CDCl₃) δ 7.40-7.15 (m, 30H), 4.82 (d, 1H), 4.62-4.43 (m, 12H), 4.38 (m, 2H), 4.29 (m, 1H), 4.22-3.95 (m, 4H), 3.91-3.60 (m, 5H), 3.54 (m, 1H), 3.42 (m, 1H), 3.25 (s, 3H), 2.09 (m, 1H), 1.87 (bs, 1H).

### Step V: Compound 2 (2R,3S,4S,5S,6R)-2-[[(2R,3S,4R,5S,6S)-3,5-Dihydroxy-2-(hydroxymethyl)-6-methoxy-tetrahydropyran-4-yl]-hydroxy-methyl]-6-(hydroxymethyl)tetrahydropyran-3,4,5-triol)

To a solution of [(2R,3S,4S,5S,6S)-3,5-Dibenzyloxy-2-(hydroxymethyl)-6-methoxy-tetrahydropyran-4-yl]-[(2R,3S,4S,5R,6R)-3,4,5-tribenzyloxy-6-(benzyloxymethyl)tetrahydropyran-2-yl]methanol (33 mg) in MeOH (3 mL) are added a catalytic amount of 20% Pd(OH)₂/C (3.9 mg, 0.03665 mmol) and a drop of acetic acid. The mixture is hydrogenated using a H₂ balloon and stirred at rt overnight. After filtration, the filtrate is concentrated to dryness and the residue is purified using reverse-phase prep-HPLC to obtain the title compound (9 mg) as a white solid.

1H NMR (400 MHz, CD₃OD) δ 4.48 (d, 1H), 4.41 (m, 1H), 4.20-4.08 (m, 1H), 4.02-3.92 (m, 2H), 3.86 (m, 3H), 3.70 (m, 4H), 3.66-3.57 (m, 1H), 3.54-3.44 (m, 1H), 3.38 (s, 3H), 1.93 (m, 1H).

LC-MS: m/z=371.0 (M+H⁺).

### Example 3

### Preparation of Compound 3 (3-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-N-methyl-benzamide)

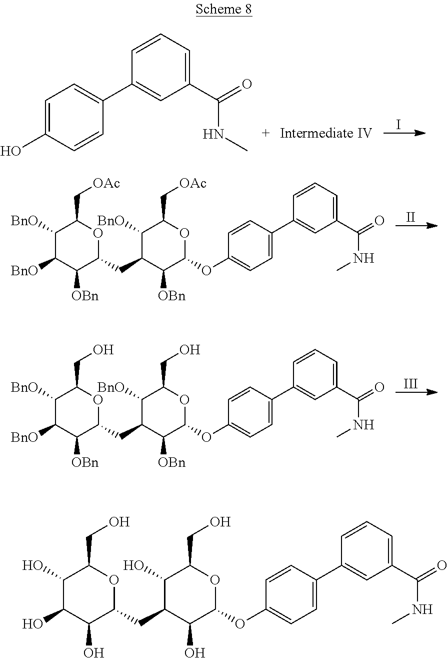

### Step I. [(2R,3R,4R,5R,6R)-6-[[(2R,3S,4S,5S,6R)-2-(Acetoxymethyl)-3,5-dibenzyloxy-6-[4-[3-(methylcarbamoyl)phenyl]phenoxy]tetrahydropyran-4-yl]methyl]-3,4,5-tribenzyloxy-tetrahydropyran-2-yl]methyl acetate

To a stirred solution containing Intermediate IV (68 mg, 0.0741 mmol) and 3-(4-hydroxyphenyl)-N-methyl-benzamide (33.7 mg, 0.1483 mmol) in DCM (3 mL) is added BF₃ etherate (18 μL, 0.1480 mmol) at 0° C. The mixture is heated to reflux under nitrogen till the starting material disappears (about 3 h). After removal of the solvent under reduced pressure, the residue is separated on Biotage™ SNAP 25 g silica gel cartridge using a gradient of ethyl acetate/hexanes 0-65% in 20 CV to obtain a mixture (42 mg), mainly containing the title compound, which is used directly in the next step without further purification.

LC-MS: m/z=1106.7 (M+Na⁺).

### Step II. 3-[4-[(2R,3S,4S,5S,6R)-3,5-Dibenzyloxy-6-(hydroxymethyl)-4-[[(2R,3R,4R,5R,6R)-3,4,5-tribenzyloxy-6-(hydroxymethyl)tetrahydropyran-2-yl]methyl]tetrahydropyran-2-yl]oxyphenyl]-N-methyl-benzamide

To a solution of the mixture containing [(2R,3R,4R,5R,6R)-6-[[(2R,3S,4S,5S,6R)-2-(acetoxymethyl)-3,5-dibenzyloxy-6-[4-[3-(methylcarbamoyl)phenyl]phenoxy]tetrahydropyran-4-yl]methyl]-3,4,5-tribenzyloxy-tetrahydropyran-2-yl]methyl acetate (42 mg) in MeOH (3 mL) is added 25% sodium methoxide (17 μL, 0.2974 mmol) and the mixture is stirred at rt overnight. It is then neutralized with resin Amberlite 120 (H). After filtration, the filtrate is concentrated to dryness to obtain a mixture (36 mg), mainly containing the title compound, which is used directly in the next step without further purification.

LC-MS: m/z=1000.7 (M+H⁺).

### Step III: Compound 3 (3-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-N-methyl-benzamide)

To a solution of 3-[4-[(2R,3S,4S,5S,6R)-3,5-dibenzyloxy-6-(hydroxymethyl)-4-[[(2R,3R,4R,5R,6R)-3,4,5-tribenzyloxy-6-(hydroxymethyl)tetrahydropyran-2-yl]methyl]tetrahydropyran-2-yl]oxyphenyl]-N-methyl-benzamide (36 mg) in MeOH (3 mL) is added a catalytic amount of 20% Pd(OH)₂/C and a drop of acetic acid. The mixture is hydrogenated using a H₂ balloon and stirred at rt overnight. After filtration, the filtrate is concentrated to dryness and the residue is purified using reverse-phase prep-HPLC to obtain the title compound (6.5 mg) as a white solid.

1H NMR (400 MHz, CD₃OD) δ 7.93 (m, 1H), 7.65 (m, 2H), 7.52 (m, 2H), 7.41 (m, 1H), 7.14 (d, 2H), 5.34 (d, 1H), 4.14-4.06 (m, 1H), 4.02 (m, 1H), 3.76-3.47 (m, 10H), 2.84 (s, 3H), 2.06 (d, 1H), 2.02-1.90 (m, 1H), 1.88-1.74 (m, 1H). LC-MS: m/z=550.5 (M+H⁺).

### Example 4

### Preparation of Compound 4 (3-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxy-3-methyl-phenyl]-N-methyl-benzamide)

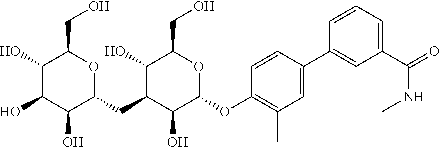 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)c(C)c2)c1]

Compound 4 (white solid, 6 mg) is prepared using a similar procedure described in Example 3.

1H NMR (400 MHz, CD₃OD) δ 7.92 (t, 1H), 7.72-7.54 (m, 2H), 7.38 (m, 3H), 7.21 (d, 1H), 5.35 (d, 1H), 4.21-4.08 (m, 1H), 4.08-3.97 (m, 1H), 3.85-3.71 (m, 2H), 3.68-3.44 (m, 8H), 2.84 (s, 3H), 2.23 (s, 3H), 2.11 (d, 1H), 2.03-1.91 (m, 1H), 1.85 (m, 1H).

LC-MS: m/z=564.5 (M+H⁺).

### Example 5

### Preparation of Compound 5 (3-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxy-N-methyl-benzamide)

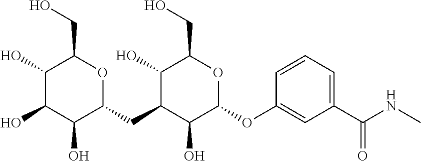 [SMILES: CNC(=O)c1cccc(O[C@H]2O[C@H](CO)[C@@H](O)[C@H](C[C@H]3O[C@H](CO)[C@@H](O)[C@H](O)[C@@H]3O)[C@@H]2O)c1]

Compound 5 (white solid, 7 mg) is prepared using a similar procedure described in Example 3.

1H NMR (400 MHz, CD₃OD) δ 7.46 (s, 1H), 7.35 (d, 1H), 7.27 (t, 1H), 7.18 (d, 1H), 5.34 (d, 1H), 4.08 (m, 1H), 4.00 (m, 1H), 3.76 (m, 2H), 3.40-3.70 (m, 8H), 2.81 (s, 3H), 2.06 (m, 1H), 1.96 (m, 1H), 1.82 (m, 1H).

LC-MS: m/z=474.5 (M+H⁺).

### Example 6

### Preparation of Compound 6 ((2R,3S,4R,5S,6R)-2-[[(2R,3S,4S,5S,6R)-3,5-Dihydroxy-2-(hydroxymethyl)-6-(3-hydroxyphenoxy)tetrahydropyran-4-yl]methyl]-6-(hydroxymethyl)tetrahydropyran-3,4,5-triol)

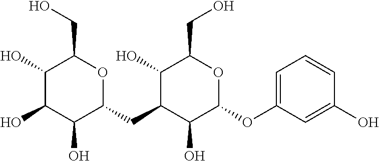 [SMILES: OC[C@H]1O[C@H](C[C@@H]2[C@H](O)[C@@H](Oc3cccc(O)c3)O[C@H](CO)[C@H]2O)[C@@H](O)[C@@H](O)[C@@H]1O]

Compound 6 (white solid, 12 mg) is prepared using a similar procedure described in Example 3.

1H NMR (400 MHz, CD₃OD) δ 6.96 (t, 1H), 6.57-6.44 (m, 2H), 6.38-6.27 (m, 1H), 5.23 (d, 1H), 4.11-3.90 (m, 2H), 3.81-3.69 (m, 2H), 3.68-3.40 (m, 8H), 2.11-1.87 (m, 2H), 1.82 (m, 1H).

LC-MS: m/z=433.4 (M+H⁺).

### Example 7

### Preparation of Compound 7 ((2R,3S,4R,5S,6R)-2-[[(2R,3S,4S,5S,6R)-3,5-Dihydroxy-2-(hydroxymethyl)-6-[4-[4-(5-methyl-1,3,4-oxadiazol-2-yl)phenyl]phenoxy]tetrahydropyran-4-yl]methyl]-6-(hydroxymethyl)tetrahydropyran-3,4,5-triol)

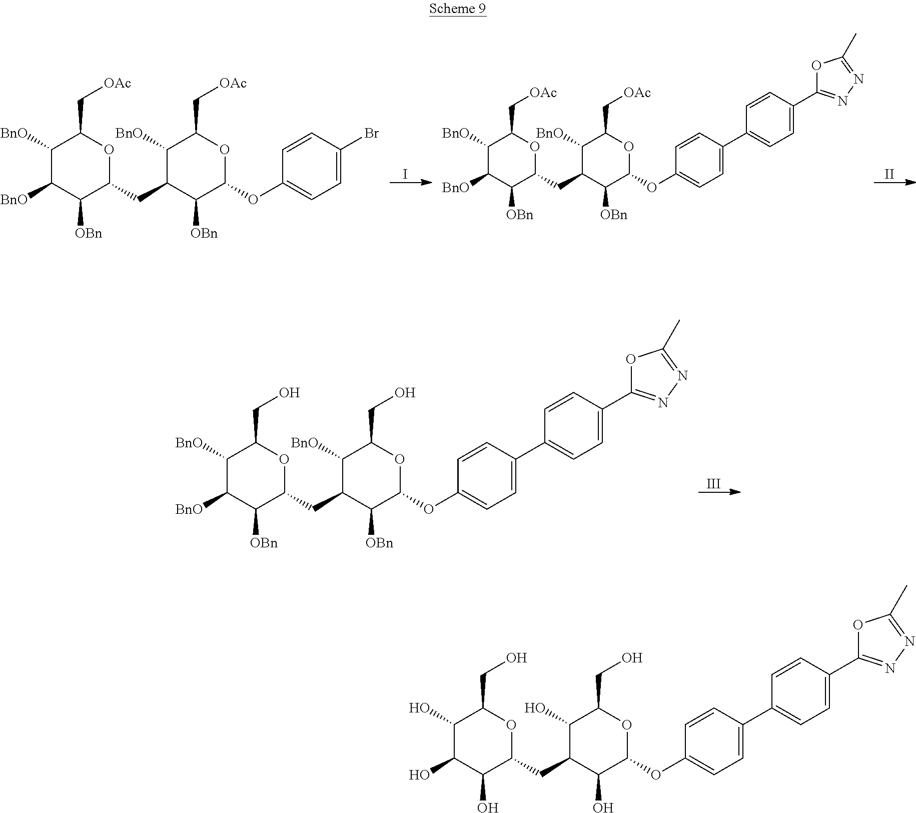

### Step I. [(2R,3R,4R,5R,6R)-6-[[(2R,3S,4S,5S,6R)-2-(Acetoxymethyl)-3,5-dibenzyloxy-6-[4-[4-(5-methyl-1,3,4-oxadiazol-2-yl)phenyl]phenoxy]tetrahydropyran-4-yl]methyl]-3,4,5-tribenzyloxy-tetrahydropyran-2-yl]methyl acetate

To a stirred solution containing Intermediate V (30 mg, 0.0291 mmol) in IPA (2 mL) are added [4-(5-methyl-1,3,4-oxadiazol-2-yl)phenyl]boronic acid (11.9 mg, 0.0583 mmol), NaHCO₃ (116.0 μL of 1 M, 0.1160 mmol) and PdCl₂(dppf)₂-DCM (2.4 mg, 0.0029 mmol). The mixture is heated to 95° C. under nitrogen and stirred for 3 h. After removal of the solvent under reduced pressure, the residue is separated on Biotage™ SNAP 10 g silica gel cartridge using a gradient of ethyl acetate/hexanes 0-50% in 20 CV to obtain a mixture (28 mg), mainly containing the title compound, which is used directly in the next step without further purification.

LC-MS: m/z=1110.0 (M+H⁺)

### Step II. [(2R,3R,4R,5R,6R)-3,4,5-Tribenzyloxy-6-[[(2R,3S,4S,5S,6R)-3,5-dibenzyloxy-2-(hydroxymethyl)-6-[4-[4-(5-methyl-1,3,4-oxadiazol-2-yl)phenyl]phenoxy]tetrahydropyran-4-yl]methyl]tetrahydropyran-2-yl]methanol

To a solution of [(2R,3R,4R,5R,6R)-6-[[(2R,3S,4S,5S,6R)-2-(acetoxymethyl)-3,5-dibenzyloxy-6-[4-[4-(5-methyl-1,3,4-oxadiazol-2-yl)phenyl]phenoxy]tetrahydropyran-4-yl]methyl]-3,4,5-tribenzyloxy-tetrahydropyran-2-yl]methyl acetate (28 mg) in MeOH (2 mL) is added a drop of 25% NaOMe (in MeOH) and the mixture is stirred at rt for 3 h. Then it is neutralized with resin Amberlite 120 (H). After filtration, the filtrate is concentrated to dryness to obtain a mixture (20 mg), mainly containing the title compound, which is used directly in the next step without further purification.

LC-MS: m/z=1025.9 (M+H⁺).

### Step III: Compound 7 ((2R,3S,4R,5S,6R)-2-[[(2R,3S,4S,5S,6R)-3,5-Dihydroxy-2-(hydroxymethyl)-6-[4-[4-(5-methyl-1,3,4-oxadiazol-2-yl)phenyl]phenoxy]tetrahydropyran-4-yl]methyl]-6-(hydroxymethyl)tetrahydropyran-3,4,5-triol)

To a solution of [(2R,3R,4R,5R,6R)-3,4,5-tribenzyloxy-6-[[(2R,3S,4S,5S,6R)-3,5-dibenzyloxy-2-(hydroxymethyl)-6-[4-[4-(5-methyl-1,3,4-oxadiazol-2-yl)phenyl]phenoxy]tetrahydropyran-4-yl]methyl]tetrahydropyran-2-yl]methanol (20 mg) in MeOH (3 mL) are added a catalytic amount of 20% Pd(OH)₂/C and a drop of acetic acid. The mixture is hydrogenated using a H₂ balloon and stirred at rt overnight. After filtration, the filtrate is concentrated to dryness and the residue is purified using reverse-phase prep-HPLC to obtain the title compound (6.5 mg) as a white solid.

1H NMR (400 MHz, CD₃OD) δ 8.04 (d, 2H), 7.76 (d, 2H), 7.63 (d, 2H), 7.23 (d, 2H), 5.44 (d, 1H), 4.16 (d, 1H), 4.10 (m, 1H), 3.83 (m, 2H), 3.52-3.75 (m, 8H), 2.62 (s, 3H), 2.15 (m, 1H), 2.05 (m, 1H), 1.92 (m, 1H).

LC-MS: m/z=575.5 (M+H⁺).

### Example 8

### Preparation of Compound 8 (5-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-N1,N3-dimethyl-benzene-1,3-dicarboxamide)

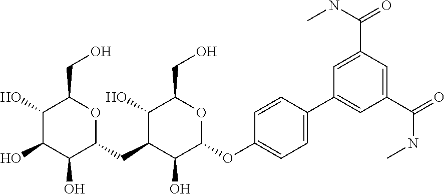 [SMILES: CNC(=O)c1cc(C(=O)NC)cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1]

Compound 8 (white solid, 2 mg) is prepared using a similar procedure described in Example 7, but using N1,N3-dimethyl-5-(4,4,5,5-tetramethyl-1,3,2-dioxaborolan-2-yl)benzene-1,3-dicarboxamide as starting material.

1H NMR (400 MHz, CD₃OD) δ 8.08 (m, 3H), 7.59 (m, 2H), 7.17 (m, 2H), 5.35 (d, 1H), 4.08 (m, 1H), 4.02 (m, 1H), 3.75 (m, 2H), 3.52-3.70 (m, 8H), 2.87 (s, 6H), 2.08 (m, 1H), 1.94 (m, 1H), 1.84 (m, 1H).

LC-MS: m/z=607.5 (M+H⁺).

### Example 9

### Preparation of Compound 9 (2-[3-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]phenyl]-N-methyl-acetamide)

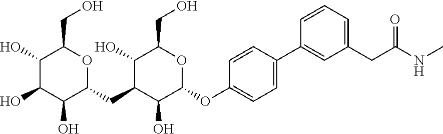 [SMILES: CNC(=O)Cc1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1]

Compound 9 (white solid, 6 mg) is prepared using a similar procedure described in Example 7, but using N-methyl-2-[3-(4,4,5,5-tetramethyl-1,3,2-dioxaborolan-2-yl)phenyl]acetamide as starting material.

1H NMR (400 MHz, CD₃OD) δ 7.52 (d, 2H), 7.48 (s, 1H), 7.42 (d, 1H), 7.33 (m, 1H), 7.20 (M, 3H), 5.41 (d, 1H), 4.11 (m, 2H), 3.82 (m, 2H), 3.52-3.70 (m, 10H), 2.87 (s, 3H), 2.15 (m, 1H), 2.04 (m, 1H), 1.92 (m, 1H).

LC-MS: m/z=564.5 (M+H⁺).

### Example 10

### Preparation of Compound 10 (2-[3-[3-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]phenyl]-N-methyl-acetamide)

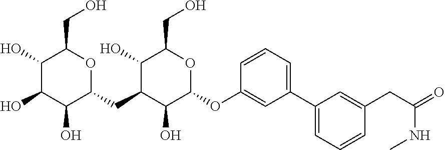 [SMILES: CNC(=O)Cc1cccc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1]

Compound 10 (white solid, 6 mg) is prepared using a similar procedure described in Example 7, but by reacting N-methyl-2-[3-(4,4,5,5-tetramethyl-1,3,2-dioxaborolan-2-yl)phenyl]acetamide with Intermediate VI.

1H NMR (400 MHz, CD₃OD) δ 7.52 (s, 1H), 7.48 (d, 1H), 7.33 (m, 3H), 7.25 (d, 2H), 7.09 (d, 1H), 5.42 (d, 1H), 4.16 (bs, 1H), 4.09 (m, 1H), 3.82 (m, 2H), 3.52-3.70 (m, 10H), 2.71 (s, 3H), 2.15 (m, 1H), 2.01 (m, 1H), 1.92 (m, 1H).

LC-MS: m/z=564.5 (M+H+).

### Example 11

### Preparation of Compound 11 (5-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-1H-pyrimidine-2,4-dione)

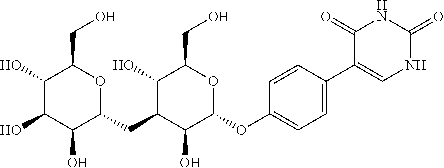 [SMILES: O=c1[nH]cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c(=O)[nH]1]

Compound 11 (white solid, 0.8 mg) is prepared using a similar procedure described in Example 7, but by reacting (2,4-ditert-butoxypyrimidin-5-yl)boronic acid with Intermediate VI, and is obtained after HPLC purification.

1H NMR (400 MHz, CD₃OD) 7.39 (s, 1H), 7.34 (d, 2H), 7.06 (d, 2H), 5.30 (d, 1H), 4.05 (m, 2H), 3.75 (m, 2H), 3.46-3.66 (m, 8H), 1.91-2.05 (m, 2H), 1.80 (m, 1H).

LC-MS: m/z=527.4 (M+H⁺).

### Example 12

### Preparation of Compound 12 (5-[4-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-6-hydroxy-3,4-dihydro-1H-pyrimidin-2-one)

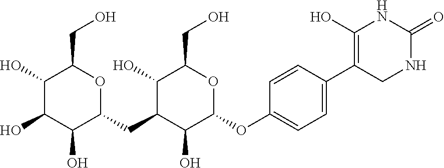 [SMILES: O=C1NCC(c2ccc(O[C@H]3O[C@H](CO)[C@@H](O)[C@H](C[C@H]4O[C@H](CO)[C@@H](O)[C@H](O)[C@@H]4O)[C@@H]3O)cc2)=C(O)N1]

Compound 12 (white solid, 1.7 mg) is prepared using a similar procedure described in Example 7, but by reacting (2,4-ditert-butoxypyrimidin-5-yl)boronic acid with Intermediate VI, and is obtained after HPLC purification.

1H NMR (400 MHz, CD₃OD) δ 7.22 (d, 2H), 7.10 (d, 2H), 5.35 (d, 1H), 4.08 (m, 2H), 3.83 (m, 3H), 3.45-3.75 (m, 9H), 1.90-2.11 (m, 2H), 1.87 (m, 1H).

LC-MS: m/z=529.5 (M+H⁺).

### Example 13

### Preparation of Compound 13 (5-[3-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-N1,N3-dimethyl-benzene-1,3-dicarboxamide)

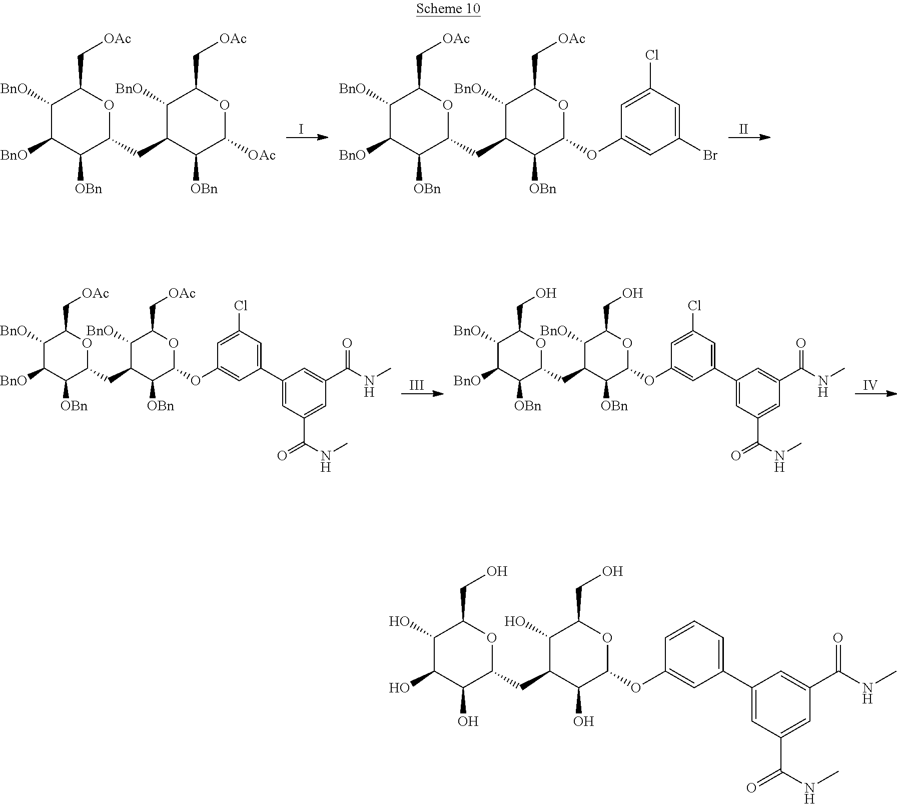

### Step I. [(2R,3R,4R,5R,6R)-6-[[(2R,3S,4S,5S,6R)-2-(Acetoxymethyl)-3,5-dibenzyloxy-6-(3-bromo-5-chloro-phenoxy)tetrahydropyran-4-yl]methyl]-3,4,5-tribenzyloxy-tetrahydropyran-2-yl]methyl acetate

To a stirred solution containing Intermediate IV (40 mg, 0.0436 mmol) and 3-bromo-5-chloro-phenol (18.10 mg, 0.0872 mmol) in DCM (3 mL) is added BF₃ etherate (11 μL, 0.0876 mmol) at rt. The mixture is stirred at rt till the starting material disappears (about 2 h). After removal of the solvent under reduced pressure, the residue is separated on Biotage™ SNAP 10 g silica gel cartridge using a gradient of ethyl acetate/hexanes 0-20% in 20 CV to obtain a mixture (27 mg), mainly containing the title compound, which is used directly in the next step without further purification.

LC-MS: m/z=1087.9 (M+Na⁺)

### Step II. [(2R,3R,4R,5R,6R)-6-[[(2R,3S,4S,5S,6R)-2-(Acetoxymethyl)-3,5-dibenzyloxy-6-[3-[3,5-bis(methylcarbamoyl)phenyl]-5-chloro-phenoxy]tetrahydropyran-4-yl]methyl]-3,4,5-tribenzyloxy-tetrahydropyran-2-yl]methyl acetate

To a stirred solution containing [(2R,3R,4R,5R,6R)-6-[[(2R,3S,4S,5S,6R)-2-(acetoxymethyl)-3,5-dibenzyloxy-6-(3-bromo-5-chloro-phenoxy)tetrahydropyran-4-yl]methyl]-3,4,5-tribenzyloxy-tetrahydropyran-2-yl]methyl acetate (30 mg, 0.0282 mmol) in IPA (2 mL) are added N1,N3-dimethyl-5-(4,4,5,5-tetramethyl-1,3,2-dioxaborolan-2-yl)benzene-1,3-dicarboxamide (13.4 mg, 0.0421 mmol), NaHCO₃ (113 μL, of 1 M, 0.113 mmol) and PdCl₂(dppf)₂-DCM (2.3 mg, 0.0028 mmol). The mixture is heated to 95° C. under nitrogen and stirred for 3 h. After removal of the solvent under reduced pressure, the residue is separated on Biotage™ SNAP 10 g silica gel cartridge using a gradient of ethyl acetate/hexanes 0-65% in 20 CV to obtain a mixture (25 mg), mainly containing the title compound which is used directly in the next step without further purification.

LC-MS: m/z=1176.9 (M+H⁺)

### Step III. 5-[3-Chloro-5-[(2R,3S,4S,5S,6R)-3,5-dibenzyloxy-6-(hydroxymethyl)-4-[[(2R,3R,4R,5R,6R)-3,4,5-tribenzyloxy-6-(hydroxymethyl)tetrahydropyran-2-yl]methyl]tetrahydropyran-2-yl]oxy-phenyl]-N1,N3-dimethyl-benzene-1,3-dicarboxamide

To a solution of the mixture (25 mg) containing [(2R,3R,4R,5R,6R)-6-[[(2R,3S,4S,5S,6R)-2-(acetoxymethyl)-3,5-dibenzyloxy-6-[3-[3,5-bis(methylcarbamoyl)phenyl]-5-chloro-phenoxy]tetrahydropyran-4-yl]methyl]-3,4,5-tribenzyloxy-tetrahydropyran-2-yl]methyl acetate in MeOH (2 mL) is added a drop of 25% NaOMe (in MeOH and the mixture is stirred at rt for 3 h. Then it is neutralized with resin Amberlite 120(H). After filtration, the filtrate is concentrated to dryness to obtain a mixture (20 mg), mainly containing the title compound, which is used directly in the next step without further purification.

LC-MS: m/z=1092.9 (M+H⁺)

### Step IV. Compound 13 (5-[3-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxyphenyl]-N1,N3-dimethyl-benzene-1,3-dicarboxamide)

To a solution of the mixture (20 mg) containing 5-[3-chloro-5-[(2R,3S,4S,5S,6R)-3,5-dibenzyloxy-6-(hydroxymethyl)-4-[[(2R,3R,4R,5R,6R)-3,4,5-tribenzyloxy-6-(hydroxymethyl)tetrahydropyran-2-yl]methyl]tetrahydropyran-2-yl]oxy-phenyl]-N1,N3-dimethyl-benzene-1,3-dicarboxamide in MeOH (3 mL) are added a catalytic amount of 20% Pd(OH)₂/C and a drop of acetic acid. The mixture is hydrogenated using a H₂ balloon and stirred at rt overnight. After filtration, the filtrate is concentrated to dryness and the residue is purified using reverse-phase prep-HPLC to obtain the title compound (2 mg) as a white solid.

1H NMR (400 MHz, CD₃OD) δ 8.15 (s, 1H), 8.12 (s, 2H), 7.43 (d, 1H), 7.34 (m, 2H), 7.11 (m, 1H), 5.38 (d, 1H), 4.10 (m, 1H), 4.03 (m, 1H), 3.73 (m, 3H), 3.45-3.72 (m, 7H), 2.86 (s, 6H), 2.10 (m, 1H), 1.92 (m, 1H), 1.85 (m, 1H).

LC-MS: m/z=607.3 (M+H⁺).

### Example 14

### Preparation of Compound 14 (2R,3S,4R,5S,6R)-2-[[(2R,3S,4S,5S,6R)-2-(1,3-Bnzodioxol-5-yloxy)-3,5-dihydroxy-6-(hydroxymethyl)tetrahydropyran-4-yl]methyl]-6-(hydroxymethyl)tetrahydropyran-3,4,5-triol

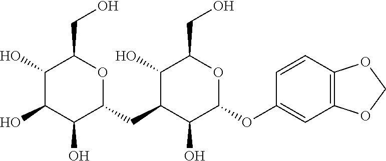 [SMILES: OC[C@H]1O[C@H](C[C@@H]2[C@H](O)[C@@H](Oc3ccc4c(c3)OCO4)O[C@H](CO)[C@H]2O)[C@@H](O)[C@@H](O)[C@@H]1O]

Compound 14 (white solid, 8 mg) is prepared using a similar procedure described in Example 3.

1H NMR (400 MHz, CD₃OD) δ 6.69-6.56 (m, 2H), 6.49 (m, 1H), 5.79 (s, 2H), 5.11 (d, 1H), 4.09-3.88 (m, 2H), 3.75 (m, 1H), 3.71-3.64 (m, 2H), 3.63-3.32 (m, 7H), 2.08-1.69 (m, 3H).

LC-MS: m/z=461.5 (M+H⁺).

### Example 15

### Preparation of Compound 15 4-[3-[(2R,3S,4S,5S,6R)-3,5-Dihydroxy-6-(hydroxymethyl)-4-[[(2R,3S,4R,5S,6R)-3,4,5-trihydroxy-6-(hydroxymethyl)tetrahydropyran-2-yl]methyl]tetrahydropyran-2-yl]oxy-4-methyl-phenyl]-N-methyl-benzamide

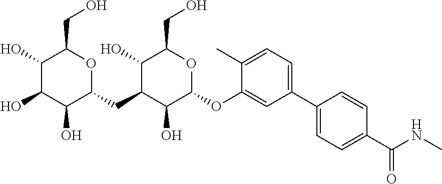 [SMILES: CNC(=O)c1ccc(-c2ccc(C)c(O[C@H]3O[C@H](CO)[C@@H](O)[C@H](C[C@H]4O[C@H](CO)[C@@H](O)[C@H](O)[C@@H]4O)[C@@H]3O)c2)cc1]

Compound 15 (white solid, 11 mg) is prepared using a similar procedure described in Example 7.

1H NMR (400 MHz, CD₃OD) δ 7.85 (d, 2H), 7.70 (d, 2H), 7.51 (s, 1H), 7.23 (t, 2H), 5.45 (d, 1H), 4.20 (s, 1H), 4.11 (d, 1H), 3.87-3.51 (m, 11H), 2.92 (s, 3H), 2.27 (s, 3H), 2.22-2.13 (m, 1H), 2.04 (m, 1H), 1.93 (m, 1H).

LC-MS: m/z=564.6 (M+H⁺).

The compounds of this invention may be prepared in light of the specification using steps generally known to those of ordinary skill in the art. Those compounds may be analyzed by known methods, including but not limited to LC-MS (liquid chromatography mass spectrometry), HPLC (high performance liquid chromatography) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein.

Mass spec. samples are analyzed on a Waters UPLC Acquity mass spectrometer operated in single MS mode with electrospray ionization. Samples are introduced into the mass spectrometer using chromatography. Mobile phase for the mass spec. analyses consisted of 0.1% formic acid and acetonitrile-water mixture. Column gradient conditions are 5%-85% acetonitrile-water over 6 minutes run time Acquity HSS T3 1.8 um 2.1 mm ID×5.0 mm. Flow rate is 1.0 mL/min. As used herein, the term “Rt(min)” refers to the LC-MS retention time, in minutes, associated with the compound. Unless otherwise indicated, the LC-MS method utilized to obtain the reported retention time is as detailed above.

Purification by reverse phase HPLC is carried out under standard conditions using a Phenomenex Gemini 21.2 mm ID×250 mm column, 5 μm, 110 Å. Elution is performed using a linear gradient CH₃CN—H₂O (with or without 0.01% TFA buffer) as mobile phase. Solvent system is tailored according to the polarity of the compound, Flow rate, 20 mL/min. Compounds are collected either by UV or Waters 3100 Mass Detector, ESI Positive Mode. Fractions containing the desired compound are combined, concentrated (rotary evaporator) to remove excess CH3CN and the resulting aqueous solution is lyophilized to afford the desired material in most cases as a white foam.

HPLC analytical method is performed on Phenomenex Gemini C18 3 um 110 Å 4.6 mm ID×250 mm, Phenomenex Gemini C18 3 um 110 Å 4.6 mm ID×50 mm, using different combinations of CH₃CN—H₂O (0.01% TFA as buffer) as mobile phase, Flow rate, 1 mL/min, PDA 210 nm. Method A: Phenomenex Gemini C18 3 um 110 Å 4.6 mm ID x 250 mm; (10-50% acetonitrile-water for 40 min, 0.01% TFA). Method B: Phenomenex Gemini C18 3 um 110 A 4.6 mm ID×250 mm; (50-90% acetonitrile-water for 40 min, 0.01% TFA). Method C: Phenomenex Gemini C18 3 um 110 A 4.6 mm ID×50 mm; (20-60% acetonitrile-water for 10 min, 0.01% TFA). Method D: Phenomenex Gemini C18 3 um 110 A 4.6 mm ID×50 mm; (10-50% acetonitrile-water for 10 min, 0.01% TFA).

### Example 14

### Competitive Binding Assay

The following competitive binding assay is performed: Round bottom, low volume 384-well microtiter plates are filled with 20 μl/well of a binding solution of FimH-CRD in 50 mM Tris, pH 7.0, 100 mM NaCl, 1 mM EDTA, 5 mM beta mercaptoethanol, 0.05% BSA, 5 nM of the Alexa647 mannoside probe and 60 nM of the FimH-CRD. Control wells using the same conditions but no FimH-CRD added are also prepared (protein-free). Compounds are serially diluted (12-point dose) in DMSO from 75 μM down to 0.4 nM. Compounds or DMSO are then added to each well (0.5 μl) to reach the final concentration of each drug (2.5% final DMSO concentration), in duplicate. Plates are then incubated for a minimum of five hours at room temperature in the dark. Plates are then read using the SpectaMax Paradigm Multi-Mode plate reader and the appropriate fluorescent polarization Detection cartridge (Alexa-647).

Alexa 647 mannoside probe is prepared using the similar procedure reported for FAM mannoside (Han, Z. et. al., 2010, J. Med. Chem., 53, 4779) and is described in the scheme below.

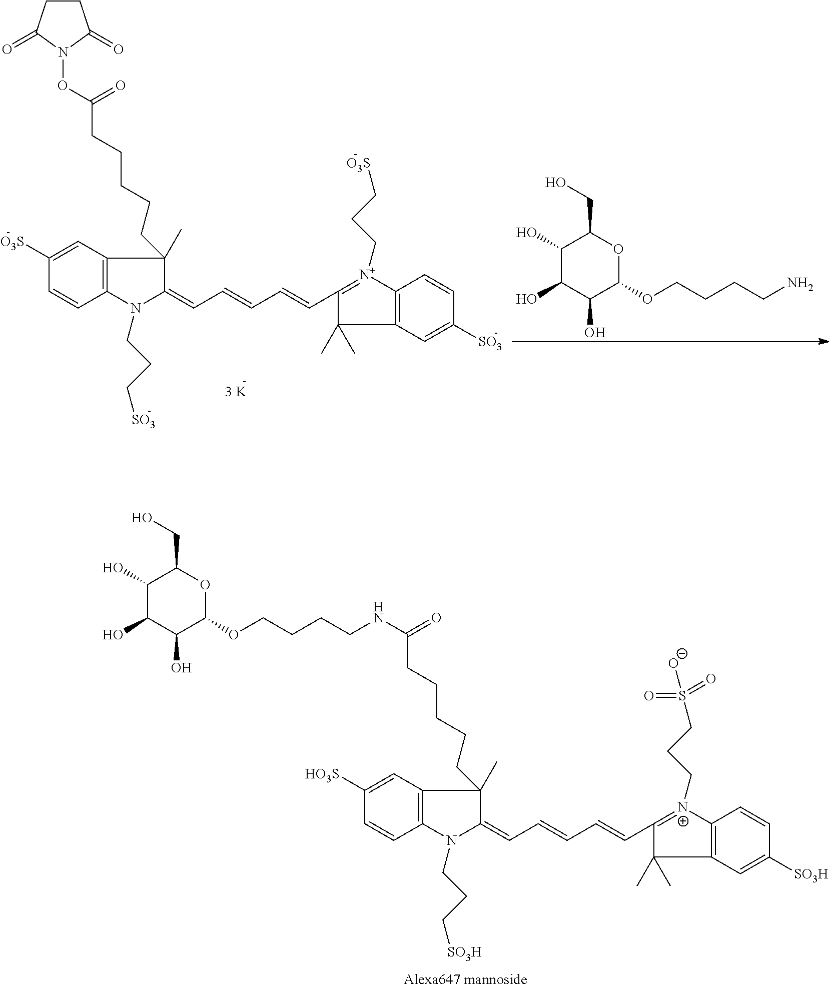

To a blue colored stirred solution of (2S,3S,4S,5S,6R)-2-(4-aminobutoxy)-6-(hydroxymethyl)tetrahydropyran-3,4,5-triol (2.21 mg, 0.009 mmol) and the (2E)-2-[(2E,4E)-5-[3,3-dimethyl-5-sulfonato-1-(3-sulfonatopropyl)indol-1-ium-2-yl]penta-2,4-dienylidene]-3-[6-(2,5-dioxopyrrolidin-1-yl)oxy-6-oxo-hexyl]-3-methyl-1-(3-sulfonatopropyl)indoline-5-sulfonate (Potassium Ion (3)) (4.9 mg, 0.0044 mmol) in DMF (44 μL) is added Et₃N (5.4 mg, 7.0 μL, 0.053 mmol) at rt. The solution is stirred at room temperature over night, concentrated, dissolved in water and purified on 12 g C-18 silica gel cartridge on Isolera system using acetonitrile in water (0 to 40%, 10 CV) and followed by lyophilization to afford Alexa 647 mannoside probe (3.3 mg, 34%) as deep blue solid.

The K_{d} values of the compounds tested in duplicates are calculated with prism software (GraphPad Software, Inc, La Jolla, USA) with Alexa-647 probe and FimH-CRD. The results are summarized in Table 1 below.

TABLE 1

| Cmpd No. | Structure | Kd(μM) |
|---|---|---|
| 1 | 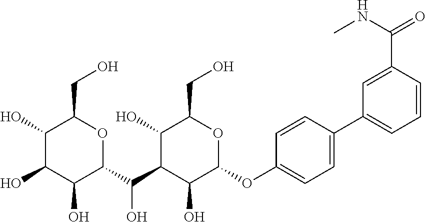 [SMILES: CNC(=O)c1cccc(-c2ccc(O[C@H]3O[C@H](CO)[C@@H](O)[C@H](C(O)[C@H]4O[C@H](CO)[C@@H](O)[C@H](O)[C@@H]4O)[C@@H]3O)cc2)c1] | 0.029 |
| 2 | 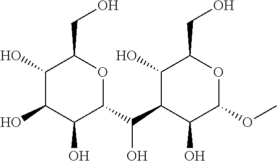 [SMILES: CO[C@H]1O[C@H](CO)[C@@H](O)[C@H](C(O)[C@H]2O[C@H](CO)[C@@H](O)[C@H](O)[C@@H]2O)[C@@H]1O] | 0.124 |
| 3 | 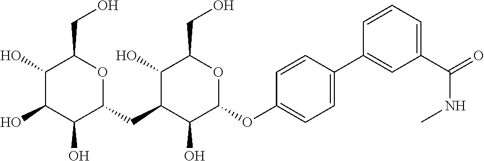 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] | 0.066 |
| 4 | 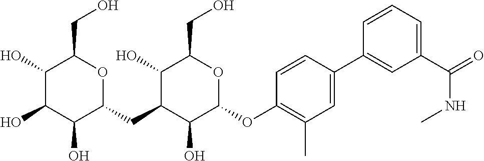 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)c(C)c2)c1] | 0.051 |
| 5 | 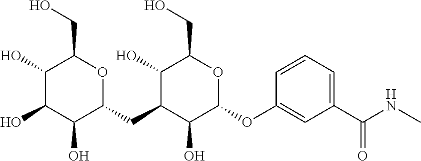 [SMILES: CNC(=O)c1cccc(O[C@H]2O[C@H](CO)[C@@H](O)[C@H](C[C@H]3O[C@H](CO)[C@@H](O)[C@H](O)[C@@H]3O)[C@@H]2O)c1] | 0.072 |
| 6 | 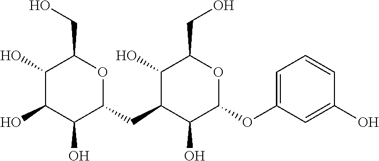 [SMILES: OC[C@H]1O[C@H](C[C@@H]2[C@H](O)[C@@H](Oc3cccc(O)c3)O[C@H](CO)[C@H]2O)[C@@H](O)[C@@H](O)[C@@H]1O] | 0.301 |
| 7 | 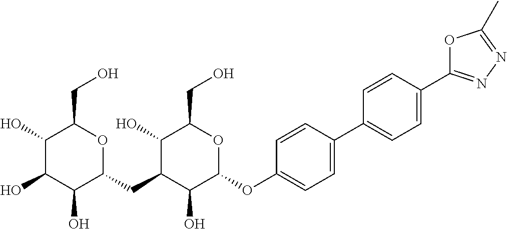 [SMILES: Cc1nnc(-c2ccc(-c3ccc(O[C@H]4O[C@H](CO)[C@@H](O)[C@H](C[C@H]5O[C@H](CO)[C@@H](O)[C@H](O)[C@@H]5O)[C@@H]4O)cc3)cc2)o1] | 0.094 |
| 8 | 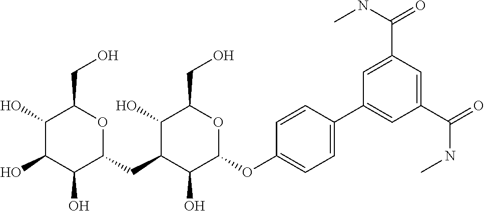 [SMILES: CNC(=O)c1cc(C(=O)NC)cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] | 0.064 |
| 9 | 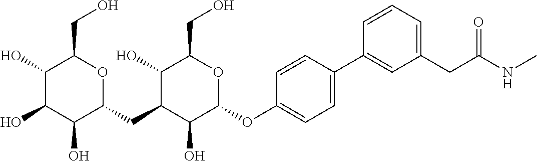 [SMILES: CNC(=O)Cc1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] | 0.079 |
| 10 | 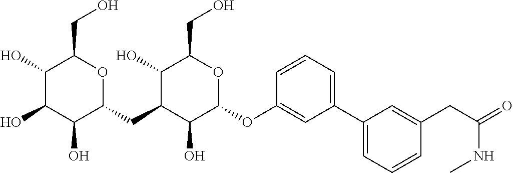 [SMILES: CNC(=O)Cc1cccc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] | 0.05 |
| 11 | 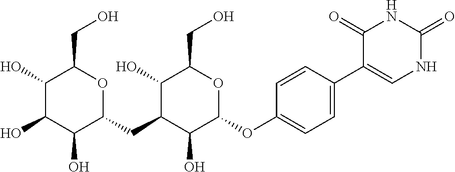 [SMILES: O=c1[nH]cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c(=O)[nH]1] | 0.097 |
| 12 | 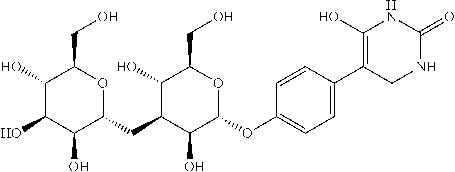 [SMILES: O=C1NCC(c2ccc(O[C@H]3O[C@H](CO)[C@@H](O)[C@H](C[C@H]4O[C@H](CO)[C@@H](O)[C@H](O)[C@@H]4O)[C@@H]3O)cc2)=C(O)N1] | 0.111 |
| 13 | 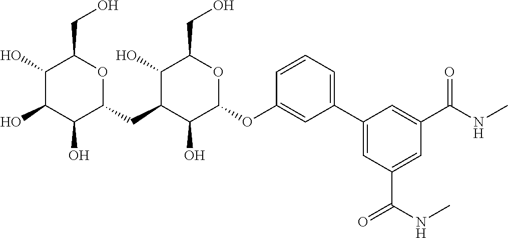 [SMILES: CNC(=O)c1cc(C(=O)NC)cc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] | 0.048 |
| 15 | 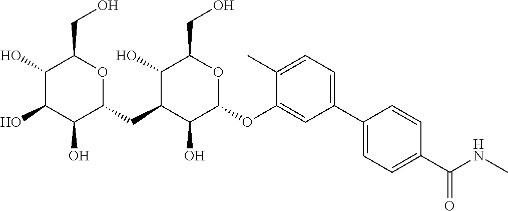 [SMILES: CNC(=O)c1ccc(-c2ccc(C)c(O[C@H]3O[C@H](CO)[C@@H](O)[C@H](C[C@H]4O[C@H](CO)[C@@H](O)[C@H](O)[C@@H]4O)[C@@H]3O)c2)cc1] | 0.019 |

While we have described a number of embodiments of this invention, it is apparent that our basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

1. A compound of Formula A; 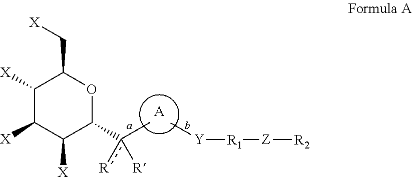
wherein:
Ring A is 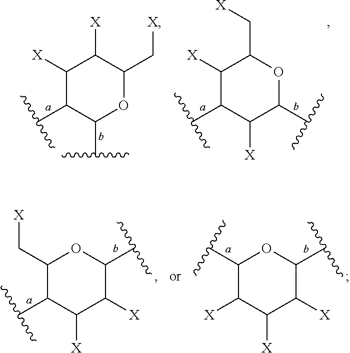
each X is independently —H, halogen, (C₁-C₆)alkyl, —NR₅R₆, —SR₇, or —OR₇;
Y and Z are each independently absent, —NR₈, —O—, or —S—;
R′ is absent, —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups;
R is —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups; wherein the dashed line represents a second bond which may be present or absent, and when present R is ═O, ═NOR₄, or ═C(R₄)₂, and R′ is absent; or
R and R′ together form a cyclic ring or a heterocyclic ring containing from 1 to 3 heteroatoms; each optionally substituted with one or more R₃ groups;
R₁ is alkyl, alkenyl, alkynyl, cycloalkyl, heterocyclyl, aryl, or heteroaryl; each optionally substituted with one or more R₃ groups;
R₂ is —H, or alkyl, alkenyl, alkynyl, cycloalkyl, heterocyclyl, aryl or heteroaryl; each optionally substituted with one or more R₃ groups, or -M-Q;
M is —C(O)O—, —C(O)—, —C(O)N(R₈)(CH₂)ₙ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈—, or (C₁-C₆)alkyl, (C₁-C₆)alkenyl or (C₁-C₆)alkynyl, wherein said (C₁-C₆)alkyl, (C₁-C₆)alkenyl or (C₁-C₆)alkynyl is optionally substituted with one or more R₃ groups;
Q is cycloalkyl, heterocyclyl, aryl or heteroaryl optionally substituted with one or more R₃ groups;
R₃ is —OH, oxo, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —C(O)NHR₄, —R₄—C(O)N(R₄)₂, —R₄—C(O)NHR₄, —N(R₄)C(O)(R₄), —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; wherein R₃ is optionally substituted with one or more R₄; and wherein each alkyl, alkenyl, alkynyl, cycloalkyl, heterocyclo, aryl, aralkyl, or heteroaryl is further optionally substituted with one or more OH or NR₇;
R₄ is —H, or optionally substituted C₁-C₆ alkyl, C₁-C₆ alkenyl, C₁-C₆ alkynyl, cycloalkyl, heterocyclyl, aryl or heteroaryl;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, —C(O)R₉, or —C(O)NHR₉, or optionally substituted alkyl, alkenyl, alkynyl, cycloalkyl, heterocyclyl, heteroaryl or aryl;
R₈ is —H, —C(O)R₉, or optionally substituted alkyl, alkenyl, alkynyl, cycloalkyl, heterocyclyl, heteroaryl, or aryl;
R₉ is —H, optionally substituted alkyl, cycloalkyl, heterocyclyl, aryl or heteroaryl;
R₁₀ is —H, halogen, or optionally substituted C₁-C₆ alkyl, C₁-C₆ alkenyl, C₁-C₆ alkynyl, cycloalkyl, heterocyclyl, aryl or heteroaryl; and
n is 0, 1, 2, 3 or 4.

2. The compound of claim 1, wherein the compound has the structure of Formula (I), (II), (III), or (IV), or a pharmaceutically acceptable salt thereof: 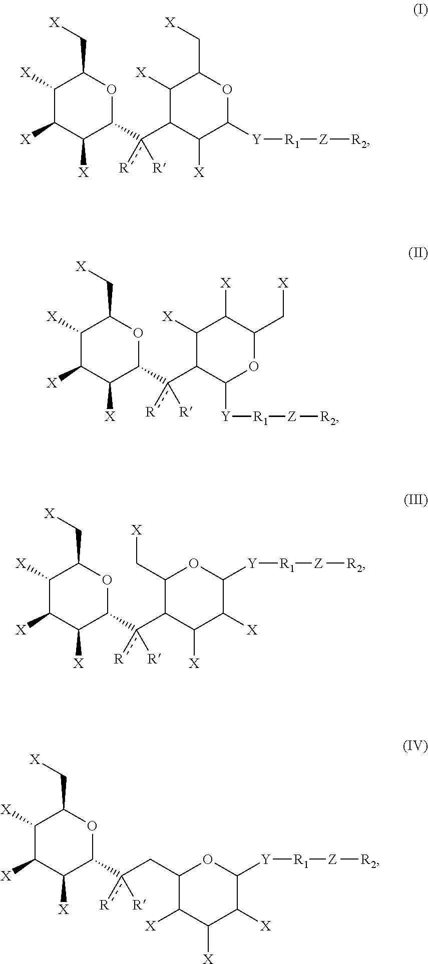
wherein:
each X is independently —H, halogen, (C₁-C₆)alkyl, —NR₅R₆, —SR₇, or —OR₇;
Y and Z are each independently absent, —NR₈, —O—, or —S—;
R′ is absent, —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups;
R is —H, halogen, —OR₄, —NR₄, —SR₄, (C₁-C₆)alkyl, (C₁-C₆)alkenyl, (C₁-C₆)alkynyl, or cycloalkyl; each optionally substituted with one or more R₃ groups; wherein the dashed line represents a second bond which may be present or absent, and when present R is ═O, ═NOR₄, or ═C(R₄)₂, and R′ is absent; or
R and R′ together form a cyclic ring or a heterocyclic ring containing from 1 to 3 heteroatoms; each optionally substituted with one or more R₃ groups;
R₁ is alkyl, alkenyl, alkynyl, cycloalkyl, heterocyclyl, aryl, or heteroaryl; each optionally substituted with one or more R₃ groups;
R₂ is —H, or alkyl, alkenyl, alkynyl, cycloalkyl, heterocyclyl, aryl or heteroaryl; each optionally substituted with one or more R₃ groups, or -M-Q;
M is —C(O)O—, —C(O)—, —C(O)N(R₈)(CH₂)ₙ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈—, or (C₁-C₆)alkyl, (C₁-C₆)alkenyl or (C₁-C₆)alkynyl, wherein said (C₁-C₆)alkyl, (C₁-C₆)alkenyl or (C₁-C₆)alkynyl is optionally substituted with one or more R₃ groups;
Q is cycloalkyl, heterocyclyl, aryl or heteroaryl optionally substituted with one or more R₃ groups;
R₃ is —OH, oxo, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —C(O)NHR₄, —R₄—C(O)N(R₄)₂, —R₄, —C(O)NHR₄, —N(R₄)C(O)(R₄), —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; wherein R₃ is optionally substituted with one or more R₄; and wherein each alkyl, alkenyl, alkynyl, cycloalkyl, heterocyclo, aryl, aralkyl, or heteroaryl is further optionally substituted with one or more OH or NR₇;
R₄ is —H, or optionally substituted C₁-C₆ alkyl, C₁-C₆ alkenyl, C₁-C₆ alkynyl, cycloalkyl, heterocyclyl, aryl or heteroaryl;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, —C(O)R₉, or —C(O)NHR₉, or optionally substituted alkyl, alkenyl, alkynyl, cycloalkyl, heterocyclyl, heteroaryl or aryl;
R₈ is —H, —C(O)R₉, or optionally substituted alkyl, alkenyl, alkynyl, cycloalkyl, heterocyclyl, heteroaryl, or aryl;
R₉ is —H, optionally substituted alkyl, cycloalkyl, heterocyclyl, aryl or heteroaryl;
R₁₀ is —H, halogen, or optionally substituted C₁-C₆ alkyl, C₁-C₆ alkenyl, C₁-C₆ alkynyl, cycloalkyl, heterocyclyl, aryl or heteroaryl; and
n is 0, 1, 2, 3 or 4.

3. The compound of claim 1, wherein the compound has the structure of Formula (I), or a pharmaceutically acceptable salt thereof:

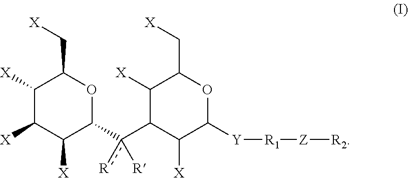

4. The compound of claim 3, or a pharmaceutically acceptable salt thereof,
wherein the Formula (I) has the following structure:

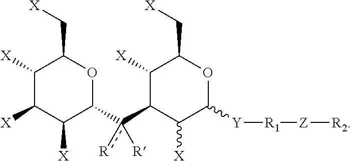

5. The compound of claim 4, or a pharmaceutically acceptable salt thereof,
wherein the Formula (I) has the following structure:

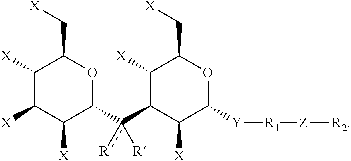

6. The compound of claim 5, wherein each X is independently —OH, —F, —OCH₃, or —CH₃, or a pharmaceutically acceptable salt thereof.

7. The compound of claim 6, wherein X is —OH; or a pharmaceutically acceptable salt thereof.

8. The compound of claim 7, or a pharmaceutically acceptable salt thereof, wherein R and R′ together form a cyclic ring or a heterocyclic ring containing from 1 to 3 heteroatoms; each optionally substituted with one or more R₃ groups.

9. The compound of claim 8, wherein R and R′ together form a 3-6 membered monocyclic cycloalkyl or heterocyclic ring containing 1-2 heteroatoms, each optionally substituted with 1-2 R₃ groups.

10. The compound of claim 9, or a pharmaceutically acceptable salt thereof, wherein the Formula (I) has the following structure:

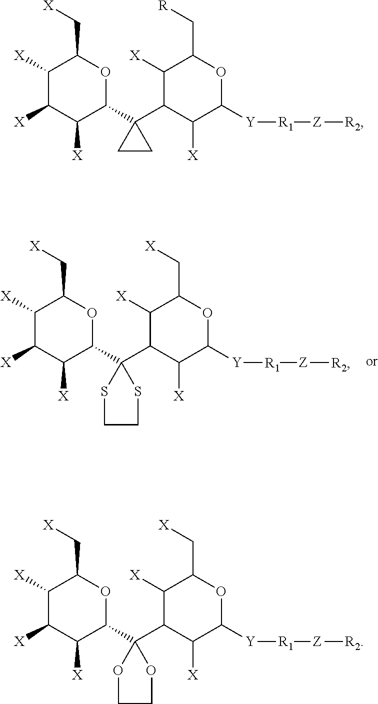

11. The compound of claim 7, wherein:
Y is —O— or —S—;
Z is absent;
R is ═O, ═NOR₄, or ═C(R₄)₂, and the dashed line representing the second bond is present;
R′ is absent;
R₁ is aryl or a 5-6 membered heterocyclyl or heteroaryl ring containing from 1 to 3 heteroatoms; each optionally substituted with one or more R₃ groups; and
R₂ is —H, aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, aryl, or heteroaryl optionally substituted with one or more R₄ groups;
wherein each R₄ is independently —H or C₁-C₆ alkyl; and
wherein n is 0, 1, or 2,
or a pharmaceutically acceptable salt thereof.

12. The compound of claim 11, or a pharmaceutically acceptable salt thereof, wherein the Formula (I) has the following structure:

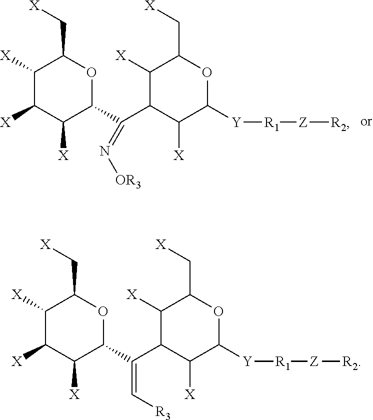

13. The compound of claim 7, wherein:
Y is —O— or —S—;
Z is absent;
R is —H, —OR₄, halogen, or (C₁-C₆)alkyl, and the dashed line representing the second bond is absent;
R′ is —H, —OR₄, halogen, or (C₁-C₆)alkyl;
R₁ is aryl or a 5-6 membered heterocyclyl or heteroaryl ring containing from 1 to 3 heteroatoms; each optionally substituted with one or more R₃ groups; and
R₂ is —H, or aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, or aryl or heteroaryl optionally substituted with one or more R₄ groups;
wherein each R₄ is independently —H or C₁-C₆ alkyl; and
wherein n is 0, 1, or 2,
or a pharmaceutically acceptable salt thereof.

14. The compound of claim 13, wherein:
Y is —O— or —S—;
Z is absent;
R is —H, —OR₄, halogen, or (C₁-C₆)alkyl, and the dashed line representing the second bond is absent;
R′ is —H, —OR₄, halogen, or (C₁-C₆)alkyl;
R₁ is aryl optionally substituted with one or more R₃ groups; and
R₂ is —H, or aryl or a 5-6 membered heterocyclyl or heteroaryl ring containing from 1 to 3 heteroatoms; each optionally substituted with one or more R₃ groups;
wherein R₃ is —OH, halogen, —(CH₂)ₙN(R₄)₂, —(CH₂)ₙC(O)N(R₄)₂, —(CH₂)ₙC(O)NHR₄, —N(R₄)C(O)(R₄)₂, —OC(O)NHR₄, —NHC(O)OR₄, —NH—C(O)R₄, —NHC(O)NHR₄, or aryl or heteroaryl optionally substituted with one or more R₄ groups;
wherein each R₄ is independently —H or C₁-C₆ alkyl; and
wherein n is 0, 1, or 2,
or a pharmaceutically acceptable salt thereof.

15. The compound of claim 14, wherein
Y is —O—;
Z is absent;
R is —H or OH and the dashed line representing the second bond is absent;
R′ is —H;
R₁ is phenyl or benzo[d][1,3]dioxolyl optionally substituted with one R₃ group;
R₂ is —H, phenyl, or a 5-6 membered heterocyclyl or heteroaryl ring containing 1 to 2 nitrogen atoms; each optionally substituted with one or more R₃ groups;
wherein R₃ is —OH, oxo, —(CH₂)ₙC(O)N(R₄)₂, C₁-C₄ alkyl, or oxadiazolyl, each optionally substituted with one or more R₄ groups;
wherein each R₄ is independently —H or C₁-C₆ alkyl; and
wherein n is 0, 1, or 2,
or a pharmaceutically acceptable salt thereof.

16. The compound of claim 15, wherein R₁ is phenyl, or a pharmaceutically acceptable salt thereof.

17. The compound of claim 16, wherein R₂ is phenyl substituted with one or more R₃ groups, or a pharmaceutically acceptable salt thereof.

18. The compound of claim 16, wherein R₃ is —OH or —(CH₂)ₙC(O)NHR₄, or a pharmaceutically acceptable salt thereof.

19. The compound of claim 18, wherein R₃ is —OH or —CH₂C(O)NHCH₃ or —C(O)NHCH₃, or a pharmaceutically acceptable salt thereof.

20. The compound of claim 16, wherein R₂ is a 5-6 membered heteroaryl ring containing from 1 to 3 nitrogen atoms; wherein the heteroaryl is optionally substituted with one or more R₃ groups, or a pharmaceutically acceptable salt thereof.

21. The compound of claim 20, wherein R₂ is a diazole optionally substituted with one or more R₃ groups, or a pharmaceutically acceptable salt thereof.

22. The compound of claim 21, wherein R₂ is a diazole optionally substituted with one or more C1-C6 alkyl groups, or a pharmaceutically acceptable salt thereof.

23. The compound of claim 22, wherein R₂ is a diazole substituted with one or more methyl groups, or a pharmaceutically acceptable salt thereof.

24. The compound of claim 16, wherein R₁ is phenyl substituted with one or more C₁-C₆ alkyl groups, or a pharmaceutically acceptable salt thereof.

25. The compound of claim 24, wherein R₁ is phenyl substituted with one or more methyl groups, or a pharmaceutically acceptable salt thereof.

26. The compound of claim 24, wherein R₂ is phenyl substituted with one or more R₃ groups, or a pharmaceutically acceptable salt thereof.

27. The compound of claim 26, wherein R₃ is —OH or —(CH₂)ₙC(O)NHR₄, or a pharmaceutically acceptable salt thereof.

28. The compound of claim 27, wherein R₃ is —OH, —CH₂C(O)NHCH₃, or —C(O)NHCH₃, or a pharmaceutically acceptable salt thereof.

29. The compound of claim 15, wherein R₁ is C₁-C₆ alkyl, or a pharmaceutically acceptable salt thereof.

30. The compound of claim 29, or a pharmaceutically acceptable salt thereof, wherein R₁ is methyl.

31. The compound of claim 29, or a pharmaceutically acceptable salt thereof, wherein R₂ is absent.

32. The compounds of claim 31, wherein Y is O.

33. The compound of claim 1, represented by a structural formula selected from the group consisting of:

| Cmpd | |
|---|---|
| No. | Structure |
| 1 | 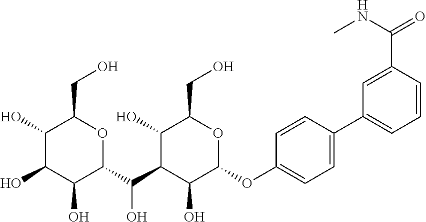 [SMILES: CNC(=O)c1cccc(-c2ccc(O[C@H]3O[C@H](CO)[C@@H](O)[C@H](C(O)[C@H]4O[C@H](CO)[C@@H](O)[C@H](O)[C@@H]4O)[C@@H]3O)cc2)c1] |
| 2 | 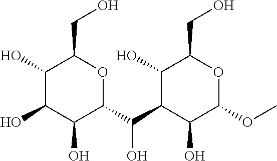 [SMILES: CO[C@H]1O[C@H](CO)[C@@H](O)[C@H](C(O)[C@H]2O[C@H](CO)[C@@H](O)[C@H](O)[C@@H]2O)[C@@H]1O] |
| 3 | 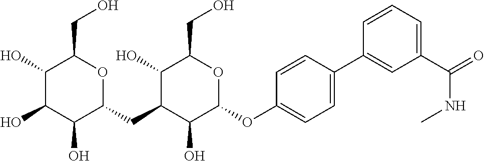 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 4 | 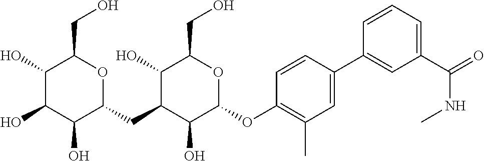 [SMILES: CNC(=O)c1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)c(C)c2)c1] |
| 5 | 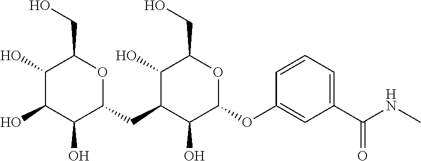 [SMILES: CNC(=O)c1cccc(O[C@H]2O[C@H](CO)[C@@H](O)[C@H](C[C@H]3O[C@H](CO)[C@@H](O)[C@H](O)[C@@H]3O)[C@@H]2O)c1] |
| 6 | 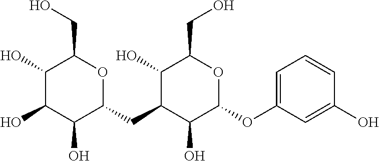 [SMILES: OC[C@H]1O[C@H](C[C@@H]2[C@H](O)[C@@H](Oc3cccc(O)c3)O[C@H](CO)[C@H]2O)[C@@H](O)[C@@H](O)[C@@H]1O] |
| 7 | 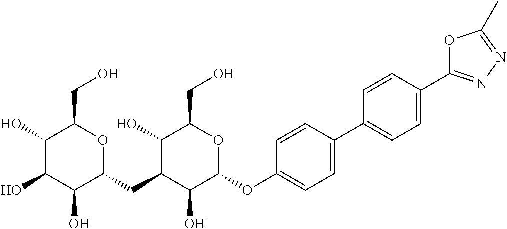 [SMILES: Cc1nnc(-c2ccc(-c3ccc(O[C@H]4O[C@H](CO)[C@@H](O)[C@H](C[C@H]5O[C@H](CO)[C@@H](O)[C@H](O)[C@@H]5O)[C@@H]4O)cc3)cc2)o1] |
| 8 | 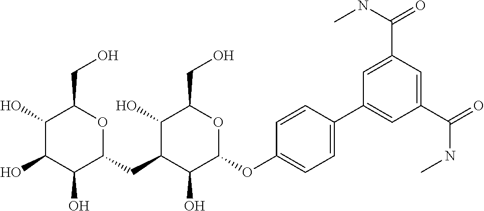 [SMILES: CNC(=O)c1cc(C(=O)NC)cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 9 | 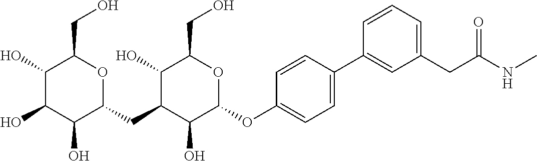 [SMILES: CNC(=O)Cc1cccc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c1] |
| 10 | 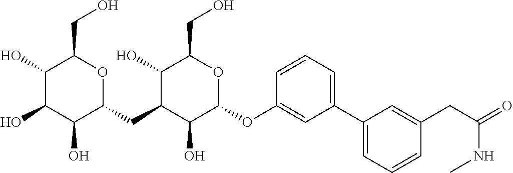 [SMILES: CNC(=O)Cc1cccc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] |
| 11 | 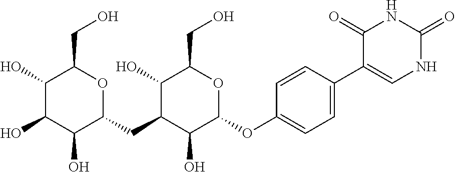 [SMILES: O=c1[nH]cc(-c2ccc(O[C@H]3O[C@H](CO)[C@@H](O)[C@H](C[C@H]4O[C@H](CO)[C@@H](O)[C@H](O)[C@@H]4O)[C@@H]3O)cc2)c(=O)[nH]1] |
| 12 | 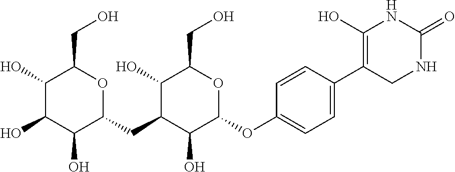 [SMILES: O=C1NCC(c2ccc(O[C@H]3O[C@H](CO)[C@@H](O)[C@H](C[C@H]4O[C@H](CO)[C@@H](O)[C@H](O)[C@@H]4O)[C@@H]3O)cc2)=C(O)N1] |
| 13 | 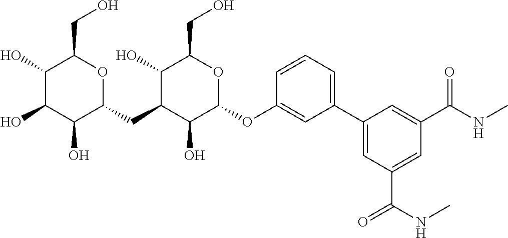 [SMILES: CNC(=O)c1cc(C(=O)NC)cc(-c2cccc(O[C@H]3O[C@H](CO)[C@@H](O)[C@H](C[C@H]4O[C@H](CO)[C@@H](O)[C@H](O)[C@@H]4O)[C@@H]3O)c2)c1] |
| 14 | 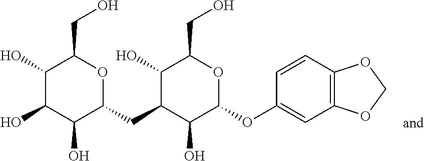 [SMILES: OC[C@H]1O[C@H](C[C@@H]2[C@H](O)[C@@H](Oc3ccc4c(c3)OCO4)O[C@H](CO)[C@H]2O)[C@@H](O)[C@@H](O)[C@@H]1O] |
| 15 | 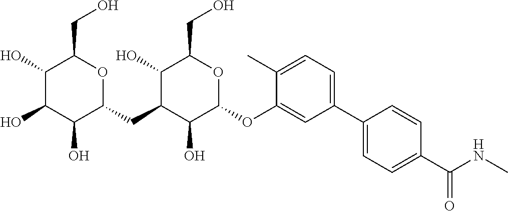 [SMILES: CNC(=O)c1ccc(-c2ccc(C)c(O[C@H]3O[C@H](CO)[C@@H](O)[C@H](C[C@H]4O[C@H](CO)[C@@H](O)[C@H](O)[C@@H]4O)[C@@H]3O)c2)cc1] |

or a pharmaceutically acceptable salt thereof.

34. A composition comprising the compound of claim 1, or a pharmaceutically acceptable salt thereof, and a pharmaceutically acceptable carrier, adjuvant, or vehicle.

35. A method of treating or preventing a bacteria infection in a subject, comprising administering to the subject an effective amount of a compound of claim 1 or a pharmaceutically acceptable salt thereof, or the composition of claim 34.

36. The method of claim 35, wherein the bacteria infection is urinary tract infection or inflammatory bowel disease.

37. A method of treating Crohn's disease or ulcerative colitis, comprising administering to the subject an effective amount of a compound of claim 1, or a pharmaceutically acceptable salt thereof, or the composition of claim 34.

38. A method of inhibiting FimH in a cell by contacting the cell with an effective amount of a compound of claim 1 or a pharmaceutically acceptable salt thereof, or the composition of claim 34.

39. A method of inhibiting adhesion or intracellular replication of AIEC in an epithelial cell by contacting the cell with an effective amount of a compound of claim 1 or a pharmaceutically acceptable salt thereof, or the composition of claim 34.

40. A method of blocking the interaction between type 1 pili and CEACAM6 in a cell by contacting the cell with an effective amount of a compound of claim 1 or a pharmaceutically acceptable salt thereof, or the composition of claim 34.
